SEQUENCE LISTING (145 sequences)

SEQ ID NO: 1 - DNA, 21 bp, Mus sp.
cagtctgctaacctggcacgt

SEQ ID NO: 2 - protein, 7 aa, Mus sp.
QSANLAR

SEQ ID NO: 3 - DNA, 21 bp, Mus sp.
caatcaggtcatctgactcgt

SEQ ID NO: 4 - protein, 7 aa, Mus sp.
QSGHLTR

SEQ ID NO: 5 - DNA, 21 bp, Mus sp.
cgttccgattcactaactaag

SEQ ID NO: 6 - protein, 7 aa, Mus sp.
RSDSLTK

SEQ ID NO: 7 - DNA, 21 bp, Mus sp.
caagctggccacctcgcttca

SEQ ID NO: 8 - protein, 7 aa, Mus sp.
QAGHLAS

SEQ ID NO: 9 - DNA, 21 bp, Mus sp.
cgttctgacaatctagcacga

SEQ ID NO: 10 - protein, 7 aa, Mus sp.
RSDNLAR

SEQ ID NO: 11 - DNA, 21 bp, Mus sp.
cgatcggataacctgcgtgaa

SEQ ID NO: 12 - protein, 7 aa, Mus sp.
RSDNLRE

SEQ ID NO: 13 - DNA, 21 bp, Mus sp.
gacagatccaaccttacccgc

SEQ ID NO: 14 - protein, 7 aa, Mus sp.
DRSNLTR

SEQ ID NO: 15 - DNA, 21 bp, Mus sp.
actacctctaaccttgctcgc

SEQ ID NO: 16 - protein, 7 aa, Mus sp.
TTSNLAR

SEQ ID NO: 17 - DNA, 21 bp, Mus sp.
cgtagtgacgctcttactcgc

SEQ ID NO: 18 - protein, 7 aa, Mus sp.
RSDALTR

SEQ ID NO: 19 - DNA, 21 bp, Mus sp.
cagtctagcaacctggcacgt

SEQ ID NO: 20 - protein, 7 aa, Mus sp.
QSSNLAR

SEQ ID NO: 21 - DNA, 21 bp, Mus sp.
cgcagcgatcatctcaccaaa

SEQ ID NO: 22 - protein, 7 aa, Mus sp.
RSDHLTK

SEQ ID NO: 23 - DNA, 21 bp, Mus sp.
caatcctctaatctcgctcgc

SEQ ID NO: 24 - protein, 7 aa, Mus sp.
QSSNLAR

SEQ ID NO: 25 - DNA, 21 bp, Homo sapiens
gaacgcggaacgctggcccgc

SEQ ID NO: 26 - protein, 7 aa, Homo sapiens
ERGTLAR

SEQ ID NO: 27 - DNA, 21 bp, Homo sapiens
gaccgctcggacttgacgcgc

SEQ ID NO: 28 - protein, 7 aa, Homo sapiens
DRSDLTR

SEQ ID NO: 29 - DNA, 21 bp, Homo sapiens
caatcctctgacttgacgcgc

SEQ ID NO: 30 - protein, 7 aa, Homo sapiens
QSSDLTR

SEQ ID NO: 31 - DNA, 21 bp, Homo sapiens
gacagatccaaccttacccgc

SEQ ID NO: 32 - protein, 7 aa, Homo sapiens
DRSNLTR

SEQ ID NO: 33 - DNA, 21 bp, Homo sapiens
cgcagcgatcatctcaccaaa

SEQ ID NO: 34 - protein, 7 aa, Homo sapiens
RSDHLTK

SEQ ID NO: 35 - DNA, 21 bp, Homo sapiens
caatcctctaatctcgctcgc

SEQ ID NO: 36 - protein, 7 aa, Homo sapiens
QSSNLAR

SEQ ID NO: 37 - DNA, 21 bp, Homo sapiens
cggagcgacaacctggctcgt

SEQ ID NO: 38 - protein, 7 aa, Homo sapiens
RSDNLAR

SEQ ID NO: 39 - DNA, 21 bp, Homo sapiens
cgcagcgatcatctcaccaaa

SEQ ID NO: 40 - protein, 7 aa, Homo sapiens
RSDHLTK

SEQ ID NO: 41 - DNA, 21 bp, Homo sapiens
gaccggagcgacctgactcgt

SEQ ID NO: 42 - protein, 7 aa, Homo sapiens
DRSDLTR

SEQ ID NO: 43 - DNA, 21 bp, Homo sapiens
gaccggagccacctgactcgt

SEQ ID NO: 44 - protein, 7 aa, Homo sapiens
DRSHLTR

SEQ ID NO: 45 - DNA, 21 bp, Homo sapiens
cggagcgacgagctgcaacgt

SEQ ID NO: 46 - protein, 7 aa, Homo sapiens
RSDELQR

SEQ ID NO: 47 - DNA, 21 bp, Homo sapiens
cggagcgaccacctgagtcgt

SEQ ID NO: 48 - protein, 7 aa, Homo sapiens
RSDHLSR

SEQ ID NO: 49 - DNA, 480 bp, Mus sp.
attggaataattggacgcaagaaagggataagtaatttgatcaaacaatttagctgttgtttttatttgtagacatcactcctgatgttgattttgggagaactggaagcttcagagggaattattaagcacagtggaagagtttcattctgctctcaattttcttggattatgccgggtactatcaaagaaaatatcatctttggtgtttcctatgatgagtacagatataagagtgttgtcaaagcttgccaactacagcaggtaagcatatttatgaaaaatgctgattgtgttagctacttgtgtcagtgttgtgataaaattgcttgactactcaccttgaaaagggttttattttaaattcttttcagggatgataccgtccatcttggcaaaggaggggcaggaatgggaagatggcgagacatgttatatccatagtcaggaagcagacagccagcaggaagtggggcttca

SEQ ID NO: 50 - DNA, 540 bp, Mus sp.
ttccagatctctgatggccattttcctcgagcctgtgcctcctctaagaacttgttggcaaaagaatgctgcccaccatggatgggtgatgggagtccctgcggccagctttcaggcagaggttcctgccaggatatccttctgtccagtgcaccatctggacctcagttccccttcaaaggggtggatgaccgtgagtcctggccctctgtgttttataataggacctgccagtgctcaggcaacttcatgggtttcaactgcggaaactgtaagtttggatttgggggcccaaattgtacagagaagcgagtcttgattagaagaaacatttttgatttgagtgtctccgaaaagaataagttcttttcttacctcactttagcaaaacatactatcagctcagtctatgtcatccccacaggcacctatggccaaatgaacaatgggtcaacacccatgtttaatgatatcaacatctacgacctctttgtatggatgcattactatgtgtcaagggacacactgcttgggggctct

SEQ ID NO: 51 - DNA, 780 bp, Homo sapiens
tgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttct

SEQ ID NO: 52 - DNA, 480 bp, Homo sapiens
gcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgtaccgggcccgccccctagcggccggggagggaggggccgggtccgcggccggcgaacggggctcgaagggtccttgtagccgggaatgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctg

SEQ ID NO: 53 - DNA, 45 bp, Artificial Sequence
  Description of Artificial Sequence Synthetic oligonucleotide
atggaaaaaccttacaagtgtccggaatgtgggaagtcctttagt

SEQ ID NO: 54 - DNA, 60 bp, Artificial Sequence
  Description of Artificial Sequence Synthetic oligonucleotide
cagcggacgcataccggtgagaagccctacaaatgcccagaatgcggaaaatcattttcg

SEQ ID NO: 55 - DNA, 60 bp, Artificial Sequence
  Description of Artificial Sequence Synthetic oligonucleotide
caacgaacccacacaggcgagaaaccatttaaatgtcctgagtgtggtaagagctttagc

SEQ ID NO: 56 - DNA, 61 bp, Artificial Sequence
  Description of Artificial Sequence Synthetic oligonucleotide
gcccgtatgagtacgttgatgnnnnnnnnnnnnnnnnnnnnngctaaagctcttaccacac

SEQ ID NO: 57 - DNA, 60 bp, Artificial Sequence
  Description of Artificial Sequence Synthetic oligonucleotide
gcctgtgtgggttcgttggtgnnnnnnnnnnnnnnnnnnnnncgaaaatgattttccgca

SEQ ID NO: 58 - DNA, 60 bp, Artificial Sequence
  Description of Artificial Sequence Synthetic oligonucleotide
accggtatgcgtccgctggtgnnnnnnnnnnnnnnnnnnnnnactaaaggacttcccaca

SEQ ID NO: 59 - DNA, 27 bp, Mus sp.
tcactcctgatgttgattttgggagaa

SEQ ID NO: 60 - DNA, 25 bp, Mus sp.
gttccccttcaaaggggtggatgac

SEQ ID NO: 61 - DNA, 30 bp, Homo sapiens
gtccccttctgggctcactatgctgccgcc

SEQ ID NO: 62 - DNA, 24 bp, Homo sapiens
ctccccggccgctagggggcgggc

SEQ ID NO: 63 - DNA, 24 bp, Mus sp.
ttccccttcaaaggggtggatgac

SEQ ID NO: 64 - DNA, 30 bp, Homo sapiens
gtcccgttcctggctcactatgctgccgcc

SEQ ID NO: 65 - DNA, 24 bp, Homo sapiens
gcccgccccctagcggccggggag

SEQ ID NO: 66 - protein, 6 aa, Artificial Sequence
  Description of Artificial Sequence Synthetic peptide
RSDETR

SEQ ID NO: 67 - protein, 6 aa, Artificial Sequence
  Description of Artificial Sequence Synthetic peptide
RSDHTT

SEQ ID NO: 68 - protein, 6 aa, Artificial Sequence
  Description of Artificial Sequence Synthetic peptide
RSDEKR

SEQ ID NO: 69 - DNA, 10 bp, Artificial Sequence
  Description of Artificial Sequence Synthetic oligonucleotide
gcgtgggcgt

SEQ ID NO: 70 - DNA, 10 bp, Artificial Sequence
  Description of Artificial Sequence Synthetic oligonucleotide
gngtgggngt

SEQ ID NO: 71 - DNA, 30 bp, Artificial Sequence
  Description of Artificial Sequence Synthetic oligonucleotide
gctctcattttccatacagtcagtatcaat

SEQ ID NO: 72 - DNA, 30 bp, Artificial Sequence
  Description of Artificial Sequence Synthetic oligonucleotide
attgatactgactgtatggaaaatgagagc

SEQ ID NO: 73 - protein, 7 aa, Artificial Sequence
  Description of Artificial Sequence Synthetic peptide
TTGNLTV

SEQ ID NO: 74 - protein, 7 aa, Artificial Sequence
  Description of Artificial Sequence Synthetic peptide
RRSACRR

SEQ ID NO: 75 - protein, 7 aa, Artificial Sequence
  Description of Artificial Sequence Synthetic peptide
HRTTLLN

SEQ ID NO: 76 - protein, 7 aa, Artificial Sequence
  Description of Artificial Sequence Synthetic peptide
DRSALAR

SEQ ID NO: 77 - protein, 7 aa, Artificial Sequence
  Description of Artificial Sequence Synthetic peptide
DASHLHT

SEQ ID NO: 78 - protein, 7 aa, Artificial Sequence
  Description of Artificial Sequence Synthetic peptide
RSDNLAR

SEQ ID NO: 79 - protein, 7 aa, Artificial Sequence
  Description of Artificial Sequence Synthetic peptide
TTGNLTV

SEQ ID NO: 80 - protein, 7 aa, Artificial Sequence
  Description of Artificial Sequence Synthetic peptide
QSGNLAR

SEQ ID NO: 81 - DNA, 21 bp, Artificial Sequence
  Description of Artificial Sequence Synthetic oligonucleotide
actacaggcaatcttacagtg

SEQ ID NO: 82 - DNA, 21 bp, Artificial Sequence
  Description of Artificial Sequence Synthetic oligonucleotide
agaaggtctgcatgtcgccgg

SEQ ID NO: 83 - DNA, 21 bp, Artificial Sequence
  Description of Artificial Sequence Synthetic oligonucleotide
catcgaacaactctacttaac

SEQ ID NO: 84 - DNA, 21 bp, Artificial Sequence
  Description of Artificial Sequence Synthetic oligonucleotide
gatcggagcgcgctagcccga

SEQ ID NO: 85 - DNA, 21 bp, Artificial Sequence
  Description of Artificial Sequence Synthetic oligonucleotide
gacgcctctcatctacacacg

SEQ ID NO: 86 - DNA, 21 bp, Artificial Sequence
  Description of Artificial Sequence Synthetic oligonucleotide
cgatcagataacttagcaagg

SEQ ID NO: 87 - DNA, 21 bp, Artificial Sequence
  Description of Artificial Sequence Synthetic oligonucleotide
accactggaaacctcacagtg

SEQ ID NO: 88 - DNA, 21 bp, Artificial Sequence
  Description of Artificial Sequence Synthetic oligonucleotide
caatcaggtaatctagcccga

SEQ ID NO: 89 - DNA, 318 bp, Artificial Sequence
  Description of Artificial Sequence Synthetic nucleotide sequence
agtggaaaaccttacaagtgtccggaatgtgggaagtcctttagtactacaggcaatcttacagtgcaccagcgtacgcatacgggagagaagccctacaaatgccccgaatgcggaaaatcattttcgagaaggtctgcatgtcgccggcaccaacggacttacaccggtgagaagccctacaaatgccccgaatgcggaaaatcattttcgcatcgaacaactctacttaaccatcaacgaacccacacaggcgagaaaccatttaaatgtcctgagtgcggtaagagctttagtgatcggagcgcgctagcccga

SEQ ID NO: 90 - DNA, 60 bp, Artificial Sequence
  Description of Artificial Sequence Synthetic oligonucleotide
attctgatgagtacgttgatgtcgggctagcgcgctccgatcactaaagctcttaccgca

SEQ ID NO: 91 - DNA, 60 bp, Artificial Sequence
  Description of Artificial Sequence Synthetic oligonucleotide
gcctgtgtgggttcgttgatggttaagtagagttgttcgatccgaaaatgattttccgca

SEQ ID NO: 92 - DNA, 60 bp, Artificial Sequence
  Description of Artificial Sequence Synthetic oligonucleotide
accggtgtaagtccgttggtgccggcgacatgcagaccttctcgaaaatgattttccgca

SEQ ID NO: 93 - DNA, 60 bp, Artificial Sequence
  Description of Artificial Sequence Synthetic oligonucleotide
tcccgtatgcgtacgctggtgcactgtaagattgcctgtagtactaaaggacttcccaca

SEQ ID NO: 94 - DNA, 318 bp, Artificial Sequence
  Description of Artificial Sequence Synthetic nucleotide sequence
atggaaaaaccttacaagtgtccggaatgtgggaagtcctttagtgacgcctctcatctacacacgcaccagcgtacgcatacgggagagaagccctacaaatgccccgaatgcggaaaatcattttcgcgatcagataacttagcaaggcaccaacggacttacaccggtgagaacccctacaaatgccccgaatgcggaaaatcattttcgaccactggaaacctcacagtgcatcaacgaacccacacaggcgagaaaccatttaaatgtcctgagtgcggtaagagctttagtcaatcaggtaatctagcccga

SEQ ID NO: 95 - DNA, 60 bp, Artificial Sequence
  Description of Artificial Sequence Synthetic oligonucleotide
attctgatgagtacgttgatgtcgggctagattacctgattgactaaagctcttaccgca

SEQ ID NO: 96 - DNA, 60 bp, Artificial Sequence
  Description of Artificial Sequence Synthetic oligonucleotide
gcctgtgtgggttcgttgatgcactgtgaggtttccagtggtcgaaaatgattttccgca

SEQ ID NO: 97 - DNA, 60 bp, Artificial Sequence
  Description of Artificial Sequence Synthetic oligonucleotide
accggtgtaagtccgttggtgccttgctaagttatctgatcgcgaaaatgattttccgca

SEQ ID NO: 98 - DNA, 60 bp, Artificial Sequence
  Description of Artificial Sequence Synthetic oligonucleotide
tcccgtatgcgtacgctggtgcgtgtgtagatgagaggcgtcactaaaggacttcccaca

SEQ ID NO: 99 - DNA, 12 bp, Artificial Sequence
  Description of Artificial Sequence Synthetic oligonucleotide
atggaaaaacct

SEQ ID NO: 100 - DNA, 12 bp, Artificial Sequence
  Description of Artificial Sequence Synthetic oligonucleotide
actcatcagaat

SEQ ID NO: 101 - DNA, 339 bp, Artificial Sequence
  CDS (1)..(339)
  Description of Artificial Sequence Synthetic nucleotide sequence
atggaaaaaccttacaagtgtccggaatgtgggaagtcctttagtactacaggcaatcttacagtgcaccagcgtacgcatacgggagagaagccctacaaatgccccgaatgcggaaaatcattttcgagaaggtctgcatgtcgccggcaccaacggacttacaccggtgagaagccctacaaatgccccgaatgcggaaaatcattttcgcatcgaacaactctacttaaccatcaacgaacccacacaggcgagaaaccatttaaatgtcctgagtgcggtaagagctttagtgatcggagcgcgctagcccgacatcaacgtactcatcagaat

SEQ ID NO: 102 - protein, 113 aa, Artificial Sequence
  Description of Artificial Sequence Synthetic protein sequence
MEKPYKCPECGKSFSTTGNLTVHQRTHTGEKPYKCPECGKSFSRRSACRRHQRTYTGEKPYKCPECGKSFSHRTTLLNHQRTHTGEKPFKCPECGKSFSDRSALARHQRTHQN

SEQ ID NO: 103 - DNA, 339 bp, Artificial Sequence
  CDS (1)..(339)
  Description of Artificial Sequence Synthetic nucleotide sequence
atggaaaaaccttacaagtgtccggaatgtgggaagtcctttagtgacgcctctcatctacacacgcaccagcgtacgcatacgggagagaagccctacaaatgccccgaatgcggaaaatcattttcgcgatcagataacttagcaaggcaccaacggacttacaccggtgagaagccctacaaatgccccgaatgtggaaaatcattttcgaccactggaaacctcacagtgcatcaacgaacccacacaggcgagaaaccatttaaatgtcctgagtgcggtaagagctttagtcaatcaggtaatctagcccgacatcaacgtactcatcagaat

SEQ ID NO: 104 - protein, 113 aa, Artificial Sequence
  Description of Artificial Sequence Synthetic protein sequence
MEKPYKCPECGKSFSDASHLHTHQRTHTGEKPYKCPECGKSFSRSDNLARHQRTYTGEKPYKCPECGKSFSTTGNLTVHQRTHTGEKPFKCPECGKSFSQSGNLARHQRTHQN

SEQ ID NO: 105 - DNA, 300 bp, Homo sapiens
gcgtgatttgataatgacctaataatgatgggttttatttccagacttcacttctaatgatgattatgggagaactggagccttcagagggtaaaattaagcacagtggaagaatttcattctgttctcagttttcctggattatgcctggcaccattaaagaaaatatcatctttggtgtttcctatgatgaatatagatacagaagcgtcatcaaagcatgccaactagaagaggtaagaaactatgtgaaaactttttgattatgcatatgaacccttcacactacccaaattatat

SEQ ID NO: 106 - DNA, 35 bp, Artificial Sequence
  Description of Artificial Sequence Synthetic oligonucleotide
atctttggtgtttcctatgatgaatatagatacag

SEQ ID NO: 107 - DNA, 12 bp, Artificial Sequence
  Description of Artificial Sequence Synthetic oligonucleotide
gacatagatata

SEQ ID NO: 108 - DNA, 12 bp, Artificial Sequence
  Description of Artificial Sequence Synthetic oligonucleotide
ataggaaacacc

SEQ ID NO: 109 - protein, 7 aa, Artificial Sequence
  Description of Artificial Sequence Synthetic peptide
DRSNLTR

SEQ ID NO: 110 - protein, 7 aa, Artificial Sequence
  Description of Artificial Sequence Synthetic peptide
QKSSLIA

SEQ ID NO: 111 - protein, 7 aa, Artificial Sequence
  Description of Artificial Sequence Synthetic peptide
TSANLSR

SEQ ID NO: 112 - protein, 7 aa, Artificial Sequence
  Description of Artificial Sequence Synthetic peptide
QKSSLIA

SEQ ID NO: 113 - protein, 7 aa, Artificial Sequence
  Description of Artificial Sequence Synthetic peptide
QKSSLIA

SEQ ID NO: 114 - protein, 7 aa, Artificial Sequence
  Description of Artificial Sequence Synthetic peptide
QSGHLQR

SEQ ID NO: 115 - protein, 7 aa, Artificial Sequence
  Description of Artificial Sequence Synthetic peptide
DSGNLRV

SEQ ID NO: 116 - protein, 7 aa, Artificial Sequence
  Description of Artificial Sequence Synthetic peptide
DKKDLTR

SEQ ID NO: 117 - DNA, 21 bp, Artificial Sequence
  Description of Artificial Sequence Synthetic oligonucleotide
gatcgctctattttgactagg

SEQ ID NO: 118 - DNA, 21 bp, Artificial Sequence
  Description of Artificial Sequence Synthetic oligonucleotide
cagaaatcttcgttgatcgca

SEQ ID NO: 119 - DNA, 21 bp, Artificial Sequence
  Description of Artificial Sequence Synthetic oligonucleotide
acttcagcgaatctttcaaga

SEQ ID NO: 120 - DNA, 21 bp, Artificial Sequence
  Description of Artificial Sequence Synthetic oligonucleotide
cagaaatcttcgttgatcgca

SEQ ID NO: 121 - DNA, 21 bp, Artificial Sequence
  Description of Artificial Sequence Synthetic oligonucleotide
cagaaatcttcgttgatcgca

SEQ ID NO: 122 - DNA, 21 bp, Artificial Sequence
  Description of Artificial Sequence Synthetic oligonucleotide
caatctgggcatctacaaagg

SEQ ID NO: 123 - DNA, 21 bp, Artificial Sequence
  Description of Artificial Sequence Synthetic oligonucleotide
gactcgggcaacctgagggta

SEQ ID NO: 124 - DNA, 21 bp, Artificial Sequence
  Description of Artificial Sequence Synthetic oligonucleotide
gacaaaaaggatttgacaaga

SEQ ID NO: 125 - DNA, 45 bp, Homo sapiens; identical to SEQ ID NO: 53

SEQ ID NO: 126 - DNA, 60 bp, Homo sapiens
cagcgtacgcatacgggagagaagccctacaaatgccccgaatgcggaaaatcattttcg

SEQ ID NO: 127 - DNA, 60 bp, Homo sapiens
caacggactcacaccggtgagaagccctacaaatgccccgaatgcggaaaatcattttcg

SEQ ID NO: 128 - DNA, 60 bp, Homo sapiens
caacgaacccacacaggcgagaaaccatttaaatgtcctgagtgcggtaagagctttagt

SEQ ID NO: 129 - DNA, 60 bp, Homo sapiens
attctgatgagtacgttgatgtgcgatcaacgaagatttctgactaaagctcttaccgca

SEQ ID NO: 130 - DNA, 60 bp, Homo sapiens
ccctgtgtgggttcgttgatgtcttgaaagattcgctgaagtcgaaaatgattttccgca

SEQ ID NO: 131 - DNA, 60 bp, Homo sapiens
accggtgtgagtccgttggtgtgcgatcaacgaagatttctgcgaaaatgattttccgca

SEQ ID NO: 132 - DNA, 60 bp, Homo sapiens
tcccgtatgcgtacgctggtgcctagtcaaattagagcgatcactaaaggacttcccaca

SEQ ID NO: 133 - DNA, 339 bp, Homo sapiens
  CDS (1)..(339)
atggaaaaaccttacaagtgtccggaatgtgggaagtcctttagtgatcgctctaatttgactaggcaccagcgtacgcatacgggagagaagccctacaaatgccccgaatgcggaaaatcattttcgcagaaatcttcgttgatcgcacaccaacggactcacaccggtgagaagccctacaaatgccccgaatgcggaaaatcattttcgacttcagcgaatctttcaagacatcaacgaacccacacaggcgagaaaccatttaaatgtcctgagtgcggtaagagctttagtcagaaatcttcgttgatcgcacatcaacgtactcatcagaat

SEQ ID NO: 134 - protein, 113 aa, Homo sapiens
MEKPYKCPECGKSFSDRSNLTRHQRTHTGEKPYKCPECGKSFSQKSSLIAHQRTHTGEKPYKCPECGKSFSTSANLSRHQRTHTGEKPFKCPECGKSFSQKSSLIAHQRTHQN

SEQ ID NO: 135 - DNA, 45 bp, Homo sapiens; identical to SEQ ID NO: 53

SEQ ID NO: 136 - DNA, 60 bp, Homo sapiens; identical to SEQ ID NO: 126

SEQ ID NO: 137 - DNA, 60 bp, Homo sapiens; identical to SEQ ID NO: 127

SEQ ID NO: 138 - DNA, 60 bp, Homo sapiens; identical to SEQ ID NO: 128

SEQ ID NO: 139 - DNA, 60 bp, Homo sapiens
attctgatgagtacgttgatgtcttgtcaaatcctttttgtcactaaagctcttaccgca

SEQ ID NO: 140 - DNA, 60 bp, Homo sapiens
gcctgtgtgggttcgttgatgtaccctcaggttgcccgagtccgaaaatgattttccgca

SEQ ID NO: 141 - DNA, 60 bp, Homo sapiens
accggtgtgagtccgttggtgcctttgtagatgcccagattgcgaaaatgattttccgca

SEQ ID NO: 142 - DNA, 60 bp, Homo sapiens
tcccgtatgcgtacgctggtgtgcgatcaacgaagatttctgactaaaggacttcccaca

SEQ ID NO: 143 - DNA, 339 bp, Homo sapiens
  CDS (1)..(339)
atggaaaaaccttacaagtgtccggaatgtgggaagtcctttagtcagaaatcttcgttgatcgcacaccagcgtacgcatacgggagagaagccctacaaatgccccgaatgcggaaaatcattttcgcaatctgggcatctacaaaggcaccaacggactcacaccggtgagaagccctacaaatgccccgaatgcggaaaatcattttcggactcgggcaacctgagggtacatcaacgaacccacacaggcgagaaaccatttaaatgtcctgagtgcggtaagagctttagtgacaaaaaggatttgacaagacatcaacgtactcatcagaat

SEQ ID NO: 144 - protein, 113 aa, Homo sapiens
MEKPYKCPECGKSFSQKSSLIAHQRTHTGEKPYKCPECGKSFSQSGHLQRHQRTHTGEKPYKCPECGKSFSDSGNLRVHQRTHTGEKPFKCPECGKSFSDKKDLTRHQRTHQN

SEQ ID NO: 145 - protein, 15 aa, Artificial Sequence
  Description of Artificial Sequence Synthetic peptide
GGGGSGGGGSGGGGS

(19) United States
(12) Patent Application Publication
(10) Pub. No.: US 2010/0055793 A1
(43) Pub. Date: Mar. 4, 2010

(54) SITE-SPECIFIC MODIFICATION OF THE HUMAN GENOME USING CUSTOM-DESIGNED ZINC FINGER NUCLEASES

(75) Inventor: Srinivasan Chandrasegaran, Baltimore, MD (US)

Correspondence Address: VENABLE LLP, P.O. BOX 34385, WASHINGTON, DC, 20043-9998, US

(73) Assignee: JOHNS HOPKINS UNIVERSITY, Baltimore, MD (US)

(21) Appl. No.: 11/989,417

(22) PCT Filed: Jul. 25, 2006
(86) PCT No.: PCT/US2006/028739

§ 371 Date: Aug. 26, 2009

Related U.S. Application Data
(60) Provisional application No. 60/702,260, filed on Jul. 25, 2005.

(51) Int. Cl. C12N 15/01; C12N 9/16

(52) U.S. Cl. 435/441; 435/196

(57) ABSTRACT

Disclosed herein are chimeric zinc finger endonucleases useful in disrupting and/or replacing at least a portion of a gene of interest (e.g. CFTR, DMPK, CCR5, TYR or βglobin).

### CROSS REFERENCE TO RELATED APPLICATIONS

The present application claims the benefit of U.S. Provisional Application No. 60/702,260, filed Jul. 25, 2005, which disclosure is hereby incorporated by reference in its entirety herein.

### TECHNICAL FIELD

The present disclosure is in the field of genome engineering.

### BACKGROUND

Molecular biologists have long sought the ability to manipulate or modify plant and mammalian genomes including the human genome at specific sites. How does one achieve targeted genome engineering of plant and mammalian cells? Cells use the universal process of homologous recombination (HR) to mediate site-specific recombination to maintain their genomic integrity, especially during the repair of a double-strand break (DSB). DSBs otherwise would be lethal to cells since they have the potential to scramble the digital information encoded within the genome of cells. DSB repair of a damaged chromosome by HR in a cell is the most accurate form of repair, which works via the copy and paste mechanism, using the homologous DNA segment from the undamaged chromosomal partner as a template. Gene targeting—the process of replacing a gene by HR—uses an extra-chromosomal fragment of donor DNA and invokes the cell's own repair machinery for gene conversion (Capecchi, 1989). Gene targeting is not a very efficient process in mammalian and plant cells; about only one in a million treated cells undergo the desired gene modification.

Molecular biologists have long known that introduction of a defined chromosomal break at a unique site within a genome, induces HR at that local site to repair the DSB (Jasin, 1996). Zinc finger nucleases (ZFNs)—proteins custom-designed to cut at specific DNA sequences—were originally developed in our lab for this purpose of delivering a targeted genomic DSB within plant and mammalian cells to enable such experiments (Kandavelou et al. 2004, 2005; Kim et al. 1996; Li et al. 1992). Reports from several labs including ours using model systems have shown that custom-designed three-finger ZFNs find and cleave their chromosomal targets in cells; and as expected, they induce local HR at the site of cleavage (Bibikova et al. 2001, 2003; Porteus & Baltimore, 2003). More recently, Urnov et al (2005) designed four-finger ZFNs that recognize an endogenous target site within the IL2Rγ gene underlying the human X-linked disease, severe combined immune deficiency (SCID) and used them for ZFN-mediated gene targeting to achieve highly efficient and permanent modification of the IL2Rγ gene in human cells.

Thus, zinc finger nuclease (ZFN)-mediated gene targeting is rapidly becoming a powerful tool for “gene editing” and “directed mutagenesis” of plant and mammalian genomes including the human genome (Kandavelou et al. 2005). ZFN-mediated gene targeting provides molecular biologists with the ability to site-specifically manipulate and permanently modify plant and mammalian genomes. Facile production of ZFNs and rapid characterization of their in vitro sequence specific cleavage properties is a pre-requisite before ZFN-mediated gene targeting can become an efficient and effective practical tool for widespread use in Biotechnology.

Here, we report the design and engineering of ZFNs that target specific endogenous sequences within mouse genes (mTYR and mCFTR) and human genes (hCCR5, hCFTR, hβglobin and hDMPK), respectively and rapid in vitro characterization of some of these ZFNs. The tested engineered ZFNs recognize their respective cognate DNA sites encoded in a plasmid substrate in a sequence-specific manner and as expected, they induce a double-strand break at the chosen target site. We also report targeted disruption of the CCR5 co-receptor in human cells by ZFN-mediated gene targeting. We have developed methods to control expression of ZFNs in mouse melanocytes to reduce cytotoxicity of ZFNs. Similar approaches could be used in plant and other mammalian cells including human cells to regulate expression of designed ZFNs in cells.

### SUMMARY

We have designed sets of ZFNs to target mouse genes, namely the tyrosinase (mTYR) and CFTR (mCFTR) and human genes, namely the CCR5 co-receptor (hCCR5) through which HIV gains entry into cells early in the infection; the DMPK gene, which is involved in myotonic dystrophy; the CFTR gene, which is involved in cystic fibrosis; and βglobin gene, involved in sickle cell anemia. Inverted sequences of the form (NNC/T)₃ or 4 . . . (G/ANN)₃ or 4 separated anywhere between 4 to 6 bp make for excellent targets for designed ZFNs without a linker. Three-finger ZFNs and four-finger ZFNs were engineered to target specific sites within these genes. The efficiency of ZFN-mediated gene targeting in vivo falls off rapidly with increasing spacer length greater than 6 bp. ZFNs with a linker are able to cleave such targets. The target sequence could be within a few hundred bp from the mutation site or the desired site of modification in the plant and mammalian genome for gene conversion.

1. We have custom-designed three-finger and four-finger ZFNs to target specific sites within mTYR and mCFTR genes of the mouse genome and hCCR5, hβglobin, hCFTR as well as hDMPK genes of the human genome, respectively.

2. These engineered ZFNs could be used for gene editing/gene correction, directed mutagenesis or for target insertion of large DNA segments (both naturally occurring DNA and synthetic DNA) at specific sites within hCCR5, hβglobin, hCFTR as well as hDMPK genes respectively by ZFN-mediated homology directed repair.

3. We have shown directed disruption of the CCR5 gene in human cells by NHEJ and by homology-directed repair.

4. Developed methods to regulate expression of ZFNs in mouse cells to reduce cytotoxicity. Similar approaches could be used to regulate expression of ZFNs in plant and human cells to reduce cytotoxicity

5. ZFPs used to engineer the ZFNs utilize consensus based framework ZF designs (Desjarlais and Berg, 1993) unlike those used by others in the field. The use of consensus framework backbone for each finger of the ZFP ensures a standard docking arrangement for each and every finger of the ZFP and hence, their mode of interaction to the DNA are very similar unlike the Zif268 based ZFPs. For these reasons, the consensus framework based ZFPs better suited for ZFN design approach compared to the ZFPs derived from Zif268 derived backbone which complicate DNA recognition.

Thus, in one aspect, described herein is a composition useful for disrupting the CCR gene in cells comprising an engineered fusion protein, said protein comprising a zinc finger binding domain to bind to a CCR5 target sequence and a cleavage domain, wherein said fusion protein binds to the CCR5 gene and cleaves the CCR5 gene.

In another aspect, described herein is a method of cleaving a CFTR gene in a cell, the method comprising: providing a fusion protein comprising a zinc finger binding domain and a Fok I cleavage domain, wherein the zinc finger binding domain binds to a target site in the CFTR gene; and contacting the cell with the fusion protein under conditions such that the CFTR gene is cleaved. In certain embodiments, the CFTR is human CFTR.

In yet another aspect, described herein is a method of cleaving a DMPK gene in a cell, the method comprising: providing a fusion protein comprising a zinc finger binding domain and a Fok I cleavage domain, wherein the zinc finger binding domain binds to a target site in the DMPK gene; and contacting the cell with the fusion protein under conditions such that the DMPK gene is cleaved.

Any of the methods described herein may further comprise the step of contacting the cell with a polynucleotide, wherein the polynucleotide replaces sequences in the cleaved CFTR gene or DMPK gene, for example replaces sequences containing mutations associated with disease (cystic fibrosis or myotonic dystrophy).

### BRIEF DESCRIPTION OF THE DRAWINGS

FIGS. 1A to D show selected ZFN target sites within the nucleotide sequences of mouse CFTR (mCFTR), mouse tyrosinase (mTYR), human CCR5 (hCCR5), and human DMPK (hDMPK) genes. The chosen targets are inverted sequences of the form (NNC)3 . . . (GNN)3 separated anywhere between 6 and 12 bp. The ZFN designs for the chosen targets that have been constructed and characterized for their DNA binding and cleavage properties are shaded. The DNA triplets adjoining the shaded ZFN target sites of the human genes for which the ZF designs are available in the literature are boxed. Other potential target sites for ZFN designs identified in the various mammalian genes are boxed. (A) Nucleotide sequence of CFTR exon 10 is shown. The site of the common CFTRΔ F508 mutation is shown in bold. (B) Nucleotide sequence of the TYR exon 1 is shown. The site of point mutation within the tyrosinase gene responsible for transition from pigmented (black) to non-pigmented (albino) mice is shown in bold. (C) Nucleotide sequence of the CCR5 gene around the (Δ32) locus is shown. The site of 32-bp deletion is shown in bold. ZFNs for the target upstream of the 32-bp region have been constructed and characterized. (D) Nucleotide sequence around the CTG triplet expansion site (in bold) of the DMPK gene is shown. The chosen ZFN target is located in the 3′untranslated region (3′ UTR) of the DMPK gene.

FIGS. 2A and 2B show synthesis of ZFP using PCR. (A) The gene for the ZFPs is first assembled using the overlapping BBOs and SDOs (60-mers) in a Klenow reaction, which is then amplified by PCR using the outside forward primer and reverse primer, which are flanked by unique restriction sites (NdeI and SpeI sites, respectively) to facilitate cloning. BBO1, BBO2, and BBO3 correspond to the consensus backbone oligos while SDO1, SDO2, and SDO3 correspond to specificity determining oligos for ZF1, ZF2, and ZF3, respectively. (B) Scheme for assembling the three-finger ZFPs via the oligo assembly strategy using the consensus framework residues and the chosen contact amino acid residues at positions −1, +1, +2, +3, +4, +5, and +6 of the α-helix, which confer specificity to each of the ZFs. The indicated top strand (bold) and bottom strand oligos overlap and will be assembled using PCR. The bottom strand oligos are depicted as having NNN, which code for the contact residues that confer specificity to each ZF.

FIGS. 3A and B depict conversion of ZFPs into ZFNs. The NdeI/SpeI-cut ZFPs are ligated into the pET-15b: N, the plasmid containing the FokI cleavage domain, with and without linker, respectively, to form pET-15b: ZFN. (A) When the inverted ZFN target sites are separated by a 4-6 bp spacer, the fusion proteins contain no linker between the ZFP and Fold cleavage domain. (B) When the inverted ZFN sites are separated by greater than 6 bp spacer (anywhere between 7 and 12 bp as in our case), the fusion proteins contain a glycine-serine linker (Gly4Ser)₃ inserted between the ZFP and FokI cleavage domain (N).

FIGS. 4A to D depict rapid in vitro characterization of the sequence specificity of the engineered ZFNs. (A) Western blot profile of the fusion proteins made using the in vitro transcription-translation (IVTT) system. This yields sufficient fusion protein for rapid characterization of the cleavage specificity of the custom-designed ZFNs. (B) Nucleotide sequences of the ZFN target sites (TS) for mCFTR, mTYR, hCCR5, and hDMPK genes, respectively, encoded in the plasmid substrates (pUC18: TS) for use in the cleavage reactions. (C) Schematic representation of the plasmid substrates (pUC18: TS) encoding the ZFN target sites for various mammalian genes at the multiple cloning site of pUC18. Four unique restriction enzyme sites namely AatII, ScaI, SspI, and XmnI within the plasmid substrates are indicated. Expected sizes of the fragments upon cleavage by ZFNs, followed by AatII, ScaI, SspI or XmnI, respectively, are shown. (D) Agarose gel profile of engineered ZFN cleavage of their respective plasmid substrates. The plasmid substrates were digested by the corresponding ZFNs, followed by one of the restriction enzymes namely AatII, SspI, ScaI or XmnI. The particular restriction enzyme used in the reactions after the corresponding ZFN digestion is indicated on top of each lane. Plasmid substrates digested with the control IVTT product (which contained no ZFN plasmids), followed by one of the enzymes, AatII, ScaI, SspI, or XmnI, respectively, for each are also shown. The 1 kb ladder marker is included in each gel profile. In the case of CCR5 gel profile, plasmid substrate cleaved using ZFN123 and ZFN456, as well as the substrate digested with either ZFN123 or ZFN456 alone, followed by ScaI restriction enzyme, is also included.

FIGS. 5A and B depict potential binding of Zif268 to other secondary sites. (A) Key base contacts deduced from the crystal structure of Zif268-DNA complex (See, also Ref. 34 in Example 1). Each finger makes contact with its target 3-bp site. In addition, Asp2 at position 2 in each finger makes contact with a base outside the 3-bp site. Fingers 1 and 3 of Zif268 make specific contacts only with two bases of their cognate DNA triplets, while base specific contacts are seen with all the three bases of finger 2. (B) Zif268 could potentially bind to other secondary sites as indicated, where N=G, A or T in top strand. All of the key base contacts shown in (A) are intact in (B).

FIGS. 6A to C depict ZFN-mediated gene targeting in human cells. (A) Targeted correction of a genetic defect by stimulating HR (recombinogenic repair) using designed ZFNs. In this experiment, cells are transfected with both ZFNs and the wild type gene or a gene fragment. (B) Targeted disruption of the CCR5 gene by NHEJ (mutagenic repair) using engineered ZFNs. Cells are transfected with ZFNs alone. CCR5 (m) depicts mutant CCR5 gene. (C) Targeted disruption of the CCR5 gene by inducing HR (recombinogenic repair) using ZFNs. In this experiment, cells are transfected with both ZFNs and CCR5Δ32 (or mutant CCR5 DNA).

FIGS. 7A and B depict targeted disruption of hCCR5 gene in human cells. (A) Cells are transfected with ZFN alone to induce mutagenic repair via NHEJ. mCCR5 indicates mutant CCR5 gene. A spectrum of different CCR5 mutant genotypes is expected from such an experiment. (B), Cells are transfected with ZFN and CCR5Δ32 (or mutant CCR5 DNA) donor DNA to induce homology-directed repair via HR. This experiment is expected to yield a single homogenous CCR5Δ32 mutant genotype.

FIGS. 8A and B depict the structure of pIRES: ZFN and pNTK7: mCCR5-Neo^{r} exogenous DNA. (A) Structure of ZFN (494-A) and ZFN (507-S); (B), Map of pNTK7: mCCR5-Neo^{r} and pIRES: ZFN-Neo(−). In pNTK7: mCCR5-GFP, the gene for Neo^{r} will be replaced with GFP, which allows for sorting the recombinant clones by flow cytometry.

FIGS. 9A to C depict flow cytometry results of ZFN transfection into CCR5 expressing Flp-In cells. (A) Isotype control. (B), CCR5 positive cells before ZFN transfection. Positive cells (>94%) are quantified in region C and negative cells (6%) in region B. (C), 3 days after ZFN transfection, 31% cells are CCR5 negative. Inset: ZFN expression in Flp-In cells post-transfection. Lanes: 1, Flp-In 293 cells before transfection; 2, 3 & 4 correspond respectively to 2, 4 and 6 days post-transfection.

FIGS. 10A and B depict positive-negative selection scheme. (A) Positive-negative selection scheme for enriching the CCR5 mutants in HEK293 cells. This protocol is similar to the one proposed by Dr. Mario Capecchi (1989) for enriching recombinants in mouse embryonic stem (ES) cells that is routinely used to make “knockout” mice. (B) Inverse PCR (IPCR) for detecting any random integration sites of the donor DNA within the genome of the mutant CCR5 HEK293 clones obtained during directed recombination by HR using ZFN and donor DNA.

FIGS. 11A to D depict a Tet-Off system for regulated expression of ZFN. (A), Scheme for creating a double-stable Tet-Off system in mouse melanocytes for controlled expression of ZEN (=Gene of interest). FIG. 14A was adapted from Clontech Tet-Off™ and Tet-On™ Gene Expression Systems User Manual. (B), Representative neomycin resistant stable cell lines of mouse melanocytes, which contain the integrated pTet-Off regulatory plasmid, were transfected with the response plasmid (pBI-Luc) encoding the luciferase gene. Cell line #5 shows a 10-fold induction of luciferase activity in absence of Dox. WD=with Dox. WOD=without Dox. (C), Structure of various plasmids to make the double-stable Tet-Off cell line; Regulatory plasmid=pTet-Off. Response plasmids=pBI-Luc or pBI: ZFN. (D), Induction of ZFN in one representative double-stable Tet-Off cell line.

FIGS. 12A to C are schematics depicting ZFNs binding to CCR. (A) shows the target sequences with bound ZF1 and ZF2. (B) depicts mutagenic repair by non-homologous end joining. (C) depicts homology-directed repair by homologous recombination.

FIG. 13 shows the binding sites for CCR5 ZFNs and ZFN amino acid and DNA sequences.

FIG. 14 shows nucleotide sequences of the CCR5 ZFN designated “CCR5 ZF 1234.”

FIG. 15 shows nucleotide sequences of the CCR5 ZFN designated “CCR5 ZF 5687.”

FIG. 16 shows amino acid sequences of the CCR5 ZFN designated “CCR5 ZF 1234.”

FIG. 17 shows amino acid sequences of the CCR5 ZFN designated “CCR5 ZF 5678.”

FIG. 18 shows a segment of the CFTR gene (exon 10, accession no. L49160) and binding sites for ZFN 1234 and ZFN 5678. Also shown are ZNF amino acid and DNA sequences.

FIG. 19 shows nucleotide sequences of the hCFTR ZFN designated “hCFTR ZF 1234.”

FIG. 20 shows amino acid sequences of the hCFTR ZFN designated “hCFTR ZF 1234.”

FIG. 21 shows nucleotide sequences of the hCFTR ZFN designated “hCFTR ZF 5678.”

FIG. 22 shows amino acid sequences of the hCFTR ZFN designated “hCFTR ZF 5678.”

### DETAILED DESCRIPTION

This invention relates, e.g., to a method for cleaving a gene of interest in a cell, the method comprising:

providing a fusion protein comprising a zinc finger binding domain and a Fok I cleavage domain, wherein the zinc finger binding domain binds to a target site in the gene of interest; and

contacting the cell with the fusion protein under conditions such that the gene of interest is cleaved.

Among the genes which can be cleaved are CFTR, DMPK, CCR5, TYR, and βglobin. Other suitable target genes will be evident to a skilled worker. The cleaved genes may be vertebrate genes, e.g. mouse, human or other mammalian genes. The cells may be from any suitable vertebrate, e.g., mammal, including mouse or human. Stem cells may be used, e.g. human or mouse adult stem cells, embryonic stem cells, or hematopoietic stem cells. Primary cells may also be used. When some genes are cleaved, specific cell types may be preferred. For example, human melanocytes or human stem cells may be used when cleaving a TYR gene. (As used herein, the term “a” includes plural referrants, e.g., can refer to two or more, unless dictated otherwise by the context in which they occur. For example, “a” TYR gene, as used above, can refer to one or more TYR genes, which can be the same or different.). For example, when CCR5 is disrupted, human or mouse primary cells, adult stem cells, embryonic stem cells or hematopoietic stem cells may be used.

A method of the invention may further comprise contacting the cell with a polynucleotide, wherein the polynucleotide replaces sequences in the cleaved gene of interest. The replaced sequences of the gene of interest may comprise at least one mutation associated with a disease or condition mediated by a mutant form of the gene of interest. For example, the following types of mutations can be replaced with wild type sequences (or, in other embodiments, the wild type sequence can be replaced with the mutant sequence): for CFTR, the mutation can be associated with cystic fibrosis; for DMPK, the mutation can be associated with muscular dystrophy; for CCR5, the mutation can associated with any function of CCR5, e.g. the ability of an HIV virus to enter a host cell via the CCR5 co-receptor; for TYR, the mutation can be associated with tyrosinase enzyme activity (e.g. related to melanin production or any of a variety of well-known neurological conditions); and for beta globin, the mutation can be associated with sickle cell anemia.

CCR 5 genes can be disrupted for a variety of purposes. For example, after cleavage of CCR5, the gene can be repaired by non-homologous end-joining in the cell to give rise to a CCR5 gene mutation that inactivates the CCR5 receptor. Alternatively, CCR5 receptor can be disrupted by replacing a wild type sequence with a CCR5delta 32 mutation. In one embodiment, a CCR5 chromosomal gene locus can serve as a “safe harbor” for the introduction of transgenes. That is, functions of CCR5 may be expendable, so that the gene can be cleaved and one of more transgenes of interest can be inserted at the cleavage site. In one embodiment, the CCR5 gene is a human gene, and one or more genes of interest can be introduced and expressed ectopically. These genes can be marker genes (e.g. neomycin or green fluorescent protein (GFP)) or genes applicable for human therapeutics.

For methods of the invention, the zinc finger domain can comprise, as a recognition region, one or more of the six 7 amino acid sequences shown in Table 1 for the listed genes. For example, the zinc finger domain may comprise three, four, or more zinc fingers. For example, in the case of a zinc finger domain for CFTR which comprises three zinc fingers, the recognition region of each of the three zinc fingers can be ZF1, ZF2 or ZF3, or it can be ZF4, ZF5 or ZF6. Other combinations, e.g. involving other genes, will be evident to the skilled worker. In some of the embodiments discussed herein, a pair of zinc finger fusion proteins is provided to a cell in order to achieve targeted cleavage, rather than a single fusion protein. As noted above, the term “a” zinc finger fusion protein, as used herein, encompasses two or more zinc finger fusion proteins.

Another aspect of the invention is a composition useful for disrupting a gene of interest in a cell (e.g., a CFTR, DMPK, CCR5, TYR, or βglobin gene) comprising an engineered fusion protein which comprises a zinc finger binding domain to bind a target sequence of the gene of interest and a FokI cleavage domain, wherein the fusion protein binds to and cleaves the gene of interest. Any of the “recognition regions” described above may be present in the fusion protein.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarnan and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

A long sought-after goal of molecular biologists has been the ability to manipulate or modify plant and mammalian genomes including the human genome at specific sites. Cells use the universal process of homologous recombination to mediate site-specific recombination and maintain their genomic integrity, particularly during the repair of a double-strand break (DSB), which otherwise would be lethal to cells. DSB repair of a damaged chromosome by homologous recombination, which works via the copy-and-paste mechanism, is the most accurate form of repair, using the homologous DNA segment from the undamaged chromosomal partner as a template. Gene targeting—the process of replacing a gene by homologous recombination—uses an extra-chromosomal fragment of donor DNA and invokes the cell's own repair machinery for gene conversion. Capecchi et al. (1989) Science 244:1288-1292. Gene targeting is not a very efficient process in mammalian cells—only about one in a million treated cells undergo the desired gene modification.

It has long been known that when a defined chromosomal break is introduced at a unique site within a genome, homologous recombination is induced at that site to repair the DSB in a large fraction of cells in a population. Jasin (1996) Trends Genet. 12:224-228. The challenge has been to develop a general means of introducing a DSB at a unique chromosomal locus in the genome to induce homology-directed repair at that site with the exogenously added donor DNA.

ZFNs—proteins custom designed to cut at specific DNA sequences—then came to the rescue. Kim et al. (1996) Proc. Nat'l Acad. Sci. USA 93: 1156-1160; Li et al. Proc. Natl. Acad. Sci. USA 89:4275-4279 (1992); Kandavelou et al. in Nucleic Acids and Molecular Biology, vol. 14 (ed. Pingoud, A. M.) 413-434 (Springer Verlag Press, Berlin, 2004). These artificial proteins combine endonuclease activity with the ability of zinc-finger domains to specifically recognize a base triplet in DNA. The Cys2H is2 zinc finger motif can target specific sequences by virtue of its unique 30 amino acid structure (stabilized by a zinc ion), the α-helix inserting into the major groove of the double helix. Amino acids within the zinc-finger motif can be changed while maintaining the remaining amino acids as a consensus backbone to generate zinc-finger motifs with new triplet sequence specificities.

Normally, three such zinc-finger domains are linked together in tandem to generate a zinc finger protein that binds to a 9-bp site, which is a composite of the individual DNA triplet subsites recognized by each of the three zinc-finger motifs. Desjarlais & Berg (1993) Proc. Natl. Acad. Sci. USA 90:2256-2260. ZFNs thus combine the nonspecific cleavage endonuclease domain of FokI restriction enzyme with zinc finger proteins to provide a general mechanism to introduce a site-specific DSB into the genome. Binding of two three-finger ZFN monomers each recognizing a 9-bp inverted site is necessary because dimerization of the Fokl cleavage domain is required to produce a DSB. Therefore, three-finger ZFNs effectively have an 18-bp recognition site, which is long enough to specify a unique address within mammalian genomes.

Reports from several laboratories using model systems have shown that designed three-finger ZFNs find and cleave their chromosomal targets in cells. As expected, they induce local homologous recombination at the site of cleavage. Bibikova et al. Mol. Cell. Biol. 21, 289-297 (2001); Porteus, M. H. & Baltimore, D. Science 300, 763 (2003); Bibikova, M., Beumer, K., Trautman, J. K. & Carroll, D. Science 300, 764 (2003).

Engineering of Chimeric Nucleases

In order to make a unique chromosomal DSB within a mammalian genome, restriction enzymes that recognize DNA sequences of 16 bp or more in length are needed. Such restriction enzyme sites occur one every 4.3×10⁹ bp on average, which is about once per human genome.

We have previously reported on chimeric nucleases including the Fok I restriction endonuclease, a bacterial Type IIS restriction enzyme. See, U.S. Pat. Nos. 6,265,196; 5,916,794; 5,792,640; and 5,487,994. FokI recognizes a nonpalindromic sequence in duplex DNA and cleaves 9/13 nucleotides downstream of the recognition site. Fold does not recognize any specific sequence at the site of cleavage. This property implies the presence of two separate protein domains within FokI: one for sequence-specific recognition of DNA and the other for endonuclease activity. Once the DNA-binding domain is anchored at the recognition site, a signal is transmitted to the other endonuclease domain, probably through allosteric interactions, and the cleavage occurs. We reasoned that one may be able to swap the FokI recognition domain with other naturally occurring DNA-binding proteins that recognize longer DNA sequences or other designed DNA binding motifs to create chimeric nucleases.

Chimeric Nucleases

The modular nature of FokI endonuclease suggested that it might be feasible to engineer chimeric nucleases by fusing other DNA-binding proteins (e.g., helix-turn-helix proteins, zinc finger proteins, helix-loop-helix proteins) to the cleavage domain of FokI. Kim and Chandrasegaran (1994) Proc. Nat'l Acad. Sci. USA 91:883-887; Kim et al. Proc. Nat'l Acad. Sci. USA (1996) 93:1156-1160.

The modular structure of zinc finger domains (ZF) and modular recognition by zinc finger proteins make them the most versatile of DNA recognition motifs for designing artificial DNA-binding proteins. Each zinc finger consists of about 30 amino acids and folds into a ββα-structure, which is stabilized by the chelation of a zinc ion by the conserved Cys2-His2 residues. Each finger typically recognizes a 3 bp DNA sequence by inserting the α-helix into the major groove of DNA. Binding of longer DNA sequences is achieved by linking several of these zinc finger motifs in tandem. Each finger, because of variations of certain key amino acids in the α-helix of one-zinc finger to the next, makes its own unique contribution to DNA-binding affinity and specificity. In theory, one can design a zinc finger for each of the 64 possible triplet codons and using a combination of these fingers, one could design a protein for sequence-specific recognition of any segment of DNA.

The creation of zinc finger chimeric nucleases (ZFN) that recognize and cleavage any target sequences depends on the reliable creation of zinc finger proteins (ZF) that can specifically recognize a target sequence. Phage display selection methods are described for example in Greisman and Pabo (1997) Science 275:657-661 and Isalan et al. (1998) Biochem 37:12-26-12033. Three different selection methods based on phage display—parallel selection, sequential selection and bipartite selection—have been reportered using Zif268-derived phage libraries for selection of designed zinc fingers. An alternative approach based on a bacterial two-hybrid system is described in Joung et al. (2000) Proc. Nat'l Acad. Sci. USA 97:7382-7387.

We are developing a double-reporter, one-hybrid system for rapidly selected zinc finger proteins and improving their sequence specificities. This system will also allow for the identification of zinc finger motifs that ct as independent modular units. This will be done by using a mutant zinc finger library that is based on consensus backbone framework for each and every finger within the protein; and by limiting the amino acid at position +2 of the α-helix of each finger to a glycine residue thus eliminating cross-strand based contact that occurs outside the 3-bp site in the Zif268 derived libraries due to the presence of Asp2.

The one-hybrid system is based on the system described in Hu et al. (2000) Methods 20:80-94. In our system, the gene coding for the zinc finger is fused to a subunit of E. coli RNA polymerase. The fusion protein is then used to activate transcription of a reporter gene under the control of a lac-derived promoter provided the zinc finger binding site is placed at an appropriate distance upstream of the promoter. Two separate operons, each containing one reporter gene under the control of a lac-derived promoter are also provided. The only difference between the two is the nature of the reporter gene and the target zinc finger binding sites, which are placed upstream of the promoter. Two different reporter systems (antibiotic resistance to chlorampenicol and tetracycline) and fluorescence (GFP, dsRED) can also be used. In this way, binding of a zinc finger protein to two different sites can be evaluated simultaneously.

Applications

In a recent issue of Nature, Urnov et al. (2005) Nature 435(7042):646-51 used four-finger zinc finger chimeric endonucleases (ZFNs) to achieve highly efficient and permanent alteration of the gene encoding human interleukin 2 receptor (IL2R), which underlies X-linked severe combined immune deficiency (SCID), commonly termed ‘bubble boy disease.’ The authors obtained a remarkable gene modification efficiency of 18% of treated cells without selection, 7% of which were altered on both X-chromosomes—a result that attests to the potential power of ZFN technology both as a research tool and in human therapeutics.

In the Nature paper, Urnov et al. add an additional finger to the ZFN design because long-term overexpression of three-finger ZFNs was shown by others to be deleterious to human cells 9. The authors posit that the additional zinc finger may confer increased specificity and selectivity to the ZFN. The resulting two four-finger ZFNs they create recognize and cut a 24-bp site in the gene encoding IL2R. The authors optimize these ZFNs for sequence-specific cleavage by tinkering with individual zinc-finger motifs in the zinc finger protein and then test the ability of the altered ZFNs to mediate correction of a mutated green fluorescent protein (GFP) gene.

ZFN optimization in HEK293 cells is achieved by monitoring gene correction frequency of a single copy of a chromosomal GFP reporter gene, which is disabled by the insertion of a fragment of IL2R gene containing the ZFN recognition sites. Several days after transient co-transfection of these GFP(−) cells with ZFN and GFP donor plasmid, FACS is used to quantify the GFP(+) cells and thereby identify the optimal ZFN. The GFP gene encoded in the donor plasmid has its first twelve base pairs and the start codon deleted to prevent its expression in cells. The donor plasmid used for in vivo gene editing contains a fragment of the IL2R locus, which is altered to carry a silent point mutation (overlaps the codon for proline at position 229) to create a novel BsrBI restriction enzyme site in exon 5. By using the optimized ZFN and the donor plasmid, Urnov et al. achieve highly efficient and permanent modification of the sequence at the endogenous IL2R locus. Thus, the sequence at the IL2R locus in human cells is altered from 5′-CCA CTC-3′ to 5′-CCG CTC-3′ by recombination with the donor plasmid. The BsrBI restriction site also overlaps the SCID missense mutation site at T703C (Leu230Pro).

Furthermore, Urnov et al. use ZFN-mediated HR to alter or correct the endogenous expression of IL2R gene in K562 cells. In a first step, they introduce a single base-pair frameshift concomitant with a DraI recognition site in exon 5 and alter IL2RA gene expression. In a second step, they restore IL2R gene expression in the mutant cells by ZFN-mediated gene editing using the donor plasmid containing the BsrBI restriction site. The ZFN-driven targeted alterations are confirmed by quantifying mRNA and protein levels in these cells.

We have identified two zinc finger target sites near the Δ32 locus of the CCR5 gene and have engineered ZFN to target and cleave one of these sites. In vitro studies indicate that the engineered ZFN bind and cleave the target site encoded in a plasmid as expected. Targeted Δ32 deletion may be induced at the chromosomal locus encoding the CCR gene in hematopoietic stem cells (CD34+ cells) of individuals who are at high risk for HIV infection. The HIV-1 resistant autologous cells are then amplified and expanded in cell culture and used to reperfuse the bone marrow of these individuals, thereby making their CD4+ lymphocytes and macrophages resistance to HIV-1 infection.

ZFN can also be designed to bind and cleave within the cystic fibrosis transmembrane conductance regulator gene (CFTR gene) so as to target cleavage and correction of the ΔF508 mutation (the most common mutation causing cystic fibrosis). Targeted correction of ΔF508 involves somatic cells.

In addition, mytonic dystrophy is yet another target. Myotonic dystrophy (DM) is the most common form of neuromuscular disease in adults, with a global incidence of 1 in 8,000 live births. It is mainly characterized by progressive muscle weakness (dystrophy) and delayed muscular relaxation (myotonia) but clinical symptoms often extend to the optic, endocrine, cardiovascular and neurological systems as well. These include ocular cataracts, type II diabetes, kidney failure, testicular atrophy, hypotesteronism and lower levels of IgM and IgG. At the same time, neurological effects manifest as cognitive impairment, hypersomnolence, hypoventilation and changes in personality and behavior. Mental retardation and development problems are associated with congenital DM, the most severe form of this disease. 30% of DMA fatalities are cause by cardiovascular disease, arising from cardiac muscle conduction defects and arrhythmias.

There are two forms of myotonic dystrophy. The most common form (DM1) is an autosomal dominant disorder linked to the myotonin gene. The second form of myotonic dystrophy (DM2) has a different genetic basis. Instead of CTG expansion, DM2 is caused by a CCTG repeat expansion in intron 1 of the zinc finger protein 9 (ZNF9) on chromosome 3. DM2 symptoms are milder and it has no severe congenital form.

The myotonin gene, which is associated with DM1, is located on the long arm of chromosome 19 (region 19q13.2), and codes for a cAMP-dependent serine-threonine kinase known as DMPK. The genetic defect of DM1 is a DNA repeat expansion in the 3′ untranslated region (UTR) of the myotonin gene. The repeat unit is a CTG triplet, and varies in number between five and several thousand. Individuals with 5-37 CTG repeats are normal and unaffected; while those with 50-80 CTG repeats are considered pre-mutations and are mildly affected or asymptomatic. 80-1000 CTG repeats in the myotonin gene causes the DM1 phenotype. Expansions of more than 1000 repeats are almost exclusively associated with congenital DMA (CDM).

A pair of ZFNs that target a specific sequence in the myotonin gene are designed and engineered.

In certain embodiments, the ZFPs are very distinct from other ZFPs because they do not use the Zif268 backbone as has been done in many other studies. Our ZFPs were designed based on the previously described zinc-finger-framework consensus sequence derived from 131 ZF sequence motifs. The specificity rules derived previously from native and mutant versions of Sp1 zinc fingers to design ZF with new specificity. All ZF domains were identical in sequence except for changes in one to four residues in its recognition region, which spans seven amino acids.

Three or more of such individual ZF motifs are linked together to form three- or more-finger proteins with different DNA-binding specificities of 9 or more bases in length. The use of consensus framework backbone for each finger of the ZFP should result in a standard docking arrangement for each and every finger and hence, their mode of interaction to the DNA is likely to be very similar unlike the Zif268 based ZFPs which is currently used by others.

Second, the oligo assembly strategy described of sequential addition of ZF motif deigns to three finger ZFPs to form four-, five- and even six-finger ZFPs.

Third, using the consensus framework based ZFPs we engineered ZFNs that target specific endogenous sequences within mouse genes (mTYR and mCFTR) and human genes (hCCR5, hCFTR, hβglobin and hDMPK), respectively. ZFNs that were tested recognize their respective cognate DNA sites encoded in a plasmid substrate in a sequence-specific manner and as expected, they induce a double-strand break at the chosen target site.

Fourth is a rapid in vitro protocol to test the sequence specific cleavage properties of these designed ZFNs.

Fifth, we used the designed consensus based ZFNs to achieve targeted disruption of CCR5 co-receptor in human cells.

Sixth, we have developed methods for regulated expression of ZFNs was achieved in mouse melanocytes to control toxicity of ZFNs. Similar approaches could be used in plant and mammalian cells to reduce cytotoxicity of ZFNs in cells. These consensus framework sequence based ZFN designs could be used for site-specific modification of the hCCR5, hCFTR, hβglobin and hDMPK genes of the human genome that is for gene editing/gene correction, directed mutagenesis and insertion of large DNA segments (large naturally occurring DNA segments as well as large synthetic DNA segment) by homology-directed repair at these gene loci. Targeted disruption of the CCR5 gene in haematopoietic stem cells could be used for human therapeutics as a form of HIV treatment by providing cells that are resistant to HIV infection in the future.

### EXAMPLES

### Example 1

### Design, Engineering and Characterization of Zinc Finger Nucleases

Recent advances in zinc finger (ZF) technology now make it possible to design and/or select ZF proteins capable of recognizing virtually any 18 bp target sequence [1], [2] and [3] long enough to specify a unique address within plant and mammalian genomes. Zinc finger nucleases (ZFNs) that combine the non-specific cleavage domain (N) of Fold restriction enzyme with ZF proteins (ZFPs), in principle, offer a general way to deliver site-specific double-strand break (DSB) within the genome [4] and [5]. The Cys2H is2 ZF proteins bind DNA by inserting an α-helix into the major groove of the double helix [6] and [7]. Each finger primarily binds to a triplet within the DNA substrate. Key amino acids at positions −1, 2, 3, and 6 relative to the start of the α-helix contribute most of the sequence-specific interactions to the ZF motifs [6] and [7]. These amino acids can be changed while maintaining the remaining amino acids as a consensus backbone to generate ZFPs with different sequence specificities [8] and [9]. The ZFP also has the additional advantage that greater specificity can be achieved by adding more ZF motifs (a maximum of six ZF domains) to the ZFPs [10], [11] and [12]. Thus, ZF DNA-binding motifs, because of their modular nature and modular structure, offer an attractive framework for designing ZFNs with tailor-made sequence-specificities [13], [14] and [15].

Several three-finger ZFPs, each recognizing a 9 bp sequence, have been fused to the non-specific endonuclease domain of FokI to form ZFNs. The cleavage specificity of ZFNs correlates directly with the binding specificity of the corresponding ZFPs that are used to make them [5] and [16]. ZFNs, like FokI restriction endonuclease [17], [18] and [19], require dimerization of the nuclease domain in order to cut DNA [20]. The dimerization of ZFNs and hence double-strand cleavage seems to be facilitated by two closely oriented inverted 9 bp binding sites [20]. Thus, ZFNs effectively have an 18 bp recognition site [20] long enough to specify a unique genomic address in plants and mammals.

Experiments from our laboratory and others using model systems have shown that ZFNs find and cleave their chromosomal targets within cells; and as expected, they induce local homologous recombination (HR) at the site of cleavage [14], [21], [22], [23] and [24]. Because DSB are lethal to the cells, in the absence of recombinogenic repair via HR (for example, when both alleles of a gene are damaged) cells repair the DSB by simple ligation via non-homologous end joining (NHEJ). Repair by NHEJ is mutagenic. Therefore, ZFNs could be used to induce “directed” mutations. This has been done in Drosophila [25] and in Arabidopsis [26]. More recently, Urnov et al. [24] have reported highly efficient and permanent modification of an endogenous gene involved in SCID in human cells using designed four-finger ZFNs. Thus, custom-designed ZFNs are becoming increasingly important as molecular tools for various biological and biomedical applications. The ability to target a DSB to a specific genomic locus and stimulate HR at that local site has great potential not only in genome engineering that is manipulation of the mammalian and plant genomes, but also in gene therapy.

Routine and facile production of ZFNs and rapid characterization of their sequence-specific cleavage properties in vitro are a pre-requisite for ZFN-mediated gene targeting to become an efficient and effective practical tool for widespread use in biological and biomedical applications. Here, we report the design, engineering, and rapid in vitro characterization of ZFNs that target specific endogenous sequences within a variety of mammalian genes. The engineered ZFNs recognize their respective DNA sites encoded in a plasmid substrate in a sequence-specific manner, and as expected, induce a DSB at the chosen target site.

Materials and Methods

The rabbit reticulocyte lysate TnTT7 Quick-Coupled Transcription-Translation system (L1170) was purchased from Promega. The restriction enzymes were from New England Biolabs (NEB). The plasmids (pUC18:TS and pET15b:ZFN) were constructed using protocols described elsewhere [16].

IVTT assay for rapid screening of ZFNs for sequence-specific cleavage activity. We have modified the IVTT assay [27] for rapidly screening the sequence-specific cleavage of the engineered ZFNs. The chosen target sites cloned into pUC18 served as the substrates [16] and [20]; the cleaved products were then analyzed by using agarose-gel electrophoresis. The designed ZFN constructs cloned into pET-15b were first transcribed and translated using the quick-coupled transcription-translation system as recommended by the manufacturer. Plasmid substrates encoding the respective ZFN target sites were then digested with 5 μl ZFN IVTT lysate or control lysate (without ZFN) for 2 h at 37° C. in NEB 4 buffer. The digest was extracted with phenol/chloroform and then precipitated with ethanol; the precipitate was air-dried and resuspended in 100 μl of autoclaved water. Ten microliters of the resuspended solution was digested with SspI in the presence of RNase A and the appropriate enzyme buffer (final volume 20 μl) at 37° C. overnight. The digest was analyzed using a 1% agarose gel. Similarly, reactions using other restriction enzymes (AatII or ScaI or XmnI, respectively) were also performed.

Results

Engineering custom-designed ZFNs for an endogenous chuomosomal gene target in mamunalian cells entails the following steps: (1) Identify target sequences of the form (NNC)3 . . . (GNN)₃ separated anywhere between 4 and 6 bp within the gene of interest, which make for excellent targets. (2) Design ZFPs that recognize a chosen target site. (3) Convert the engineered ZFPs into ZFNs. (4) Rapidly characterize their in vitro cleavage specificity, which is essential before any in vivo studies can be performed using the designed ZFNs.

### Step 1: Selection of ZFN Target Sites within Various Mammalian Genes

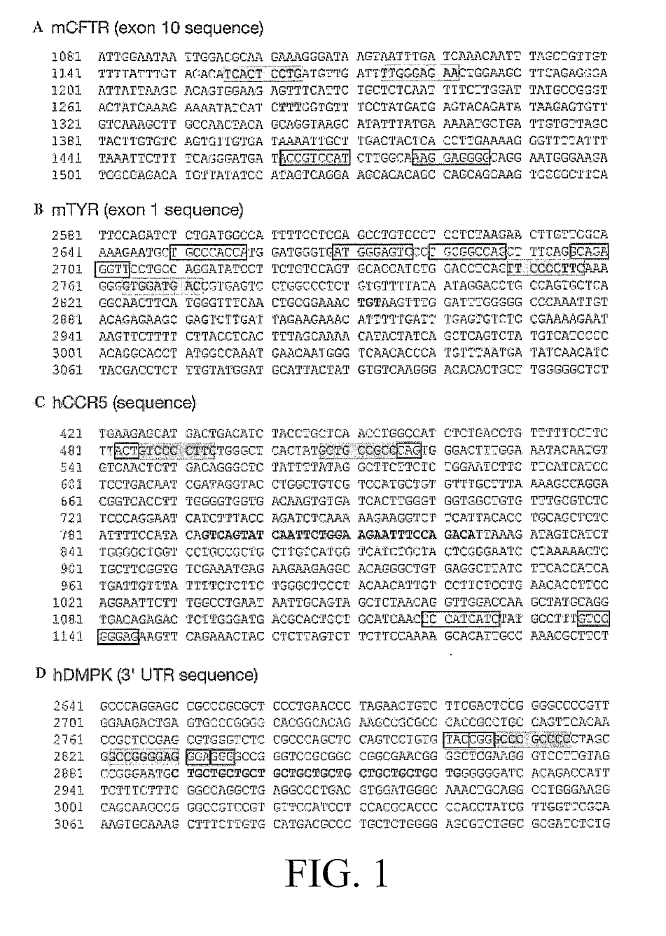

As part of this study, we have designed sets of three-finger ZFNs to target each of the two mouse genes, namely the tyrosinase (mTYR) and CFTR (mCFTR), and each of the two human genes, namely the CCR5 co-receptor (hCCR5) through which HIV gains entry into cells early in the infection and the DMPK gene, which is involved in myotonic dystrophy. Inverted sequences of the form (NNC)3 . . . (GNN)3 separated anywhere between 4 and 6 bp make for excellent targets. The efficiency of ZFN-mediated gene targeting in vivo falls off rapidly with increasing spacer length beyond 6 bp. The target sequence could be within a few hundred base pair from the mutation site for gene conversion. The ZFN target sites for the mTYR and mCFTR genes (FIG. 1) were provided to us by Casey Case and Ed Rebar of Sangamo BioSciences.

The ZFN targets for the human genes were identified (by simple eye inspection) looking for (NNC)3 . . . (GNN)3 within a few hundred base pair sequence flanking the mutation sites (both at the 3′ and 5′ ends) of the human genes. These are depicted in FIG. 1. In many instances, more than one ZFN target sites with different spacer lengths were identified.

### Step 2: ZFP Design and Construction

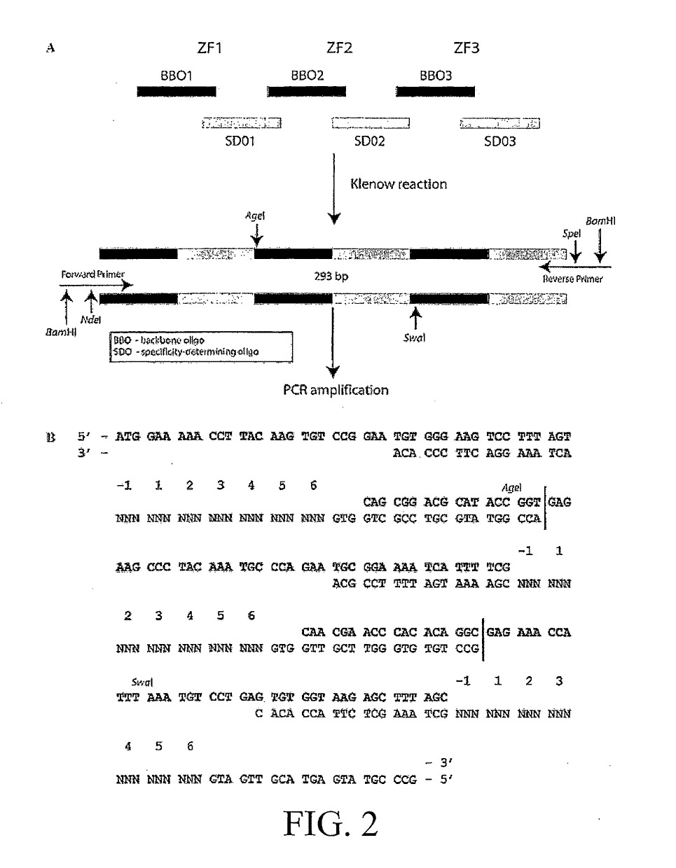

The ZFPs discussed in this article are very distinct from other ZFPs because they do not use the Zif268 backbone as has been done in many other studies. Our ZFPs were designed based on the previously described zinc-finger-framework consensus sequence derived from 131 ZF sequence motifs [8]. Berg's laboratory combined the consensus backbone framework sequence with specificity rules derived from native and mutant versions of Sp1 ZF motifs to design ZFPs with new specificity. All of the ZF motifs within the three-finger ZFPs were essentially identical in their amino acid sequence, except for changes in their recognition region, which spans about seven amino acids of the α-helix. Three such individual ZF motifs are then linked together to form three-zinc-finger proteins with different DNA-binding specificities of 9 bp in length. We designed ZFPs that recognize a specific 9 bp sequence within the chosen mammalian genes as follows: (1) By using the consensus framework backbone sequence for each and every finger within the ZFPs using three invariant amino acid backbone oligos (BBO1, BBO2, and BBO3). (2) By varying the contact residues at positions −1, +1, +2, +3, +4, +5, and +6 of the α-helix within each ZF using three specificity determining oligos (SDO1, SDO2, and SDO3); the amino acid residues that confer specificity were chosen from previously available DNA triplet recognition data for ZFPs in the literature [28], [29], [30] and [31] and wherever possible taking into account the positional data of each ZF motif in the context of its neighboring fingers (FIG. 2; Table 1). (3) By placing unique restriction sites between each of the fingers to enable selective replacement of individual fingers with other ZF motifs to generate three-finger ZFPs with new sequence specificities (FIG. 2). This construct also allows for increasing the number of ZF motifs within the ZFPs, as and when needed, by adding more ZF motifs to the N-terminal or the C-terminal end of the ZFPs, provided the ZF designs that recognize the adjoining triplets of the target site are known. The use of consensus framework backbone for each finger of the ZFP should result in a standard docking arrangement for each and every finger and hence, their mode of interaction to the DNA is likely to be very similar.

TABLE 1

ZF designs for the chosen targets within various mammalian
genes

| | | | DNA coding sequence/contact |
|---|---|---|---|
| | ZFN | Triplet | residues (−1 to +6 positions) of |
| | target site^{a} | subsites^{a} | the α-helix for the ZF designs |
| Gene | 5′-3′ | 5′-3′ | −1 +1 +2 +3 +4 +5 +6 |
| mCFTR | TTG GGA GAA c | ZF1 GAA c | CAG TCT GCT AAC CTG GCA GGT |
| | | | Q S A N L A R |
| | | ZF2 GGA g | CAA TCA GGT CAT CTG ACT CGT |
| | | | Q S G H L T R |
| | | ZF3 TTG g | CGT TCC GAT TCA CTA ACT AAG |
| | | | R S D S L T K |
| | CAG GAG TGA t | ZF4 TGA t | CAA GCT GGC CAC CTC GCT TCA |
| | | | Q A G H L A S |
| | | ZF5 GAG t | CGT TCT GAC AAT CTA GCA CGA |
| | | | R S D N L A R |
| | | ZF6 CAG g | CGA TCG GAT AAC CTG CGT GAA |
| | | | R S D N L R E |
| mTYR | GTG GAT GAC c | ZF1 GAC c | GAC AGA TCC AAC CTT ACC CGC |
| | | | D R S N L T R |
| | | ZF2 GAT g | ACT ACC TCT AAC CTT GCT CGC |
| | | | T T S N L A R |
| | | ZF3 GTG g | CGT AGT GAC GCT CTT ACT CGC |
| | | | R S D A L T R |
| | GAA GGG GAA g | ZF4 GAA g | CAG TCT AGC AAC CTG GCA CGT |
| | | | Q S S N L A R |
| | | ZF5 GGG g | CGC AGC GAT CAT CTC ACC AAA |
| | | | R S D H L T K |
| | | ZF6 GAA g | CAA TCC TCT AAT CTC GCT CGC |
| | | | Q S S N L A R |
| hCCR5 | GCT GCC GCC c | ZF1 GCC c | GAA CGC GGA ACG CTG GCC CGC |
| | | | E R G T L A R |
| | | ZF2 GCC g | GAC CGC TCG GAC TTG ACG CGC |
| | | | D R S D L T R |
| | | ZF3 GCT g | CAA TCC TCT GAC TTG ACG CGC |
| | | | Q S S D L T R |
| | GAA GGG GAC a | ZF4 GAC a | GAC AGA TCC AAC CTT ACC CGC |
| | | | D R S N L T R |
| | | ZF5 GGG g | CGC AGC GAT CAT CTC ACC AAA |
| | | | R S D H L T K |
| | | ZF6 GAA g | CAA TCC TCT AAT CTC GCT CGC |
| | | | Q S S N L A R |
| hDMPK | GCC GGG GAG g | ZF1 GAG g | CGG AGC GAC AAC CTG GCT CGT |
| | | | R S D N L A R |
| | | ZF2 GGG g | CGC AGC GAT CAT CTC ACC AAA |
| | | | R S D H L T K |
| | | ZF3 GCC g | GAC CGG AGC GAC CTG ACT CGT |
| | | | D R S D L T R |
| | GGG GCG GGC c | ZF4 GGC c | GAC CGG AGC CAC CTG ACT CGT |
| | | | D R S H L T R |
| | | ZF5 GCG g | CGG AGC GAC GAG CTG CAA CGT |
| | | | R S D E L Q R |
| | | ZF6 GGG g | CGG AGC GAC CAC CTG AGT CGT |
| | | | R S D H L S R |

^{a}The base 3′ to the chosen 9 bp targets and DNA subsites is shown in lowercase type.

The ZFN designs for the target sites within the various mammalian genes are shown in Table 1. The DNA coding sequence for the contact residues at positions −1 to +6 of the α-helix is also included. The overlapping oligo assembly strategy was used to construct the three-finger ZFPs (FIG. 2A). They were first assembled by Klenow reaction using the BBOs and SDOs (FIG. 2B). The assembled three-finger ZFPs were then amplified by PCR using the forward primer (flanked by a NdeI site) and reverse primer (flanked by a SpeI site) to facilitate cloning of the engineered ZFPs.

### Step 3: Converting Designed ZFPs into ZFNs

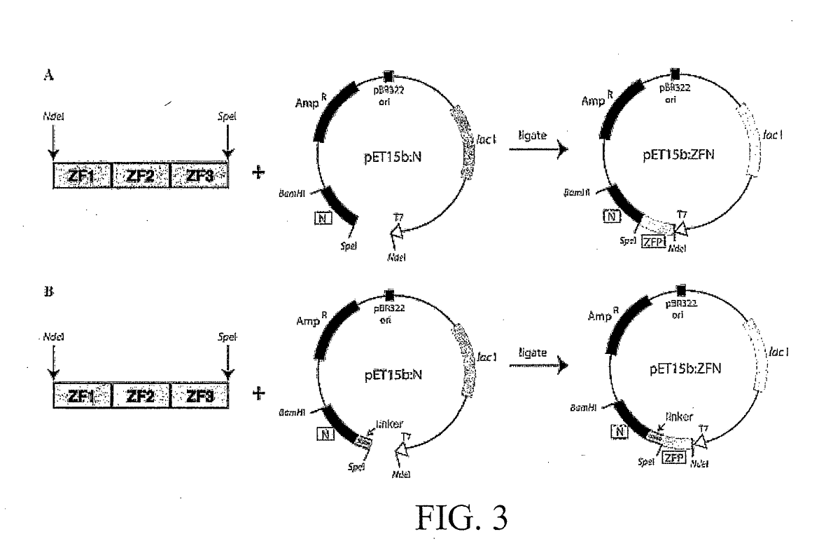

The PCR-amplified DNA coding for the ZFPs was digested with NdeI/SpeI and then ligated into the NdeI/SpeI-cleaved pET-15b: ZFN vector, thereby replacing the existing ZFPs with the newly generated ZFPs. These constructs link the consensus framework based ZFPs to the C-terminal 196 amino acids of FokI restriction enzyme, which constitutes the FokI cleavage domain (FIG. 3A). The ZFN fusions are of the form “NH3+-ZF1-ZF2-ZF3-FokI (N)—CO2-.” When the separation between the ZFN target sites is 4-6 bp which are optimal for efficient cleavage, no linker is included between the ZFPs and FokI cleavage domain; however, for ZFN targets with greater than 6 bp separation, the ZFP is connected to the Fold cleavage domain through a (Gly4Ser)3 linker (FIG. 3B). Furthermore, during the initial cloning of the engineered ZFNs into the bacterial cells, clones carrying the ZFN constructs are made more viable by increasing the levels of the DNA ligase within these cells [5] and [16].

### Step 4: Rapid Characterization of the Designed ZFNs for Sequence-Specific Cleavage

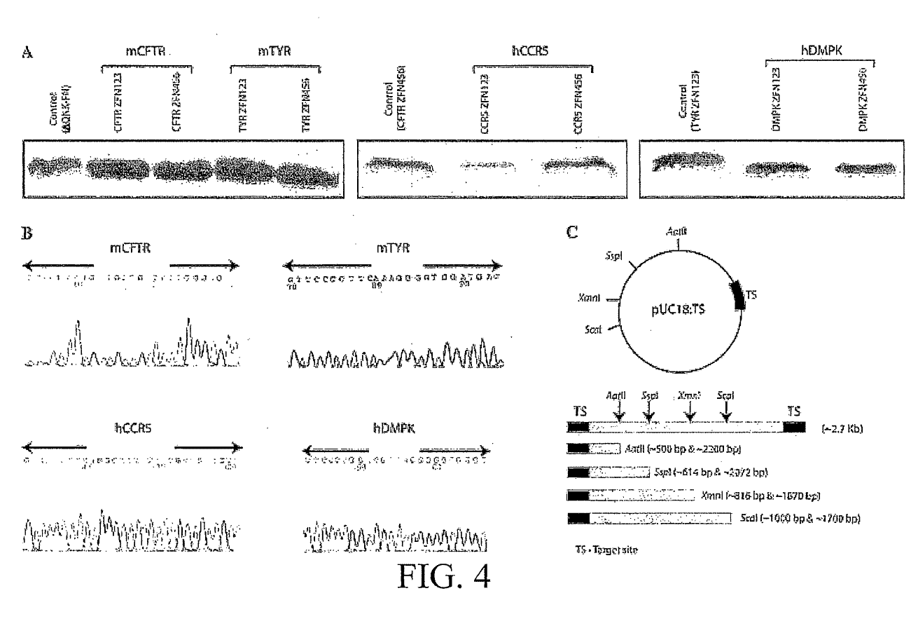

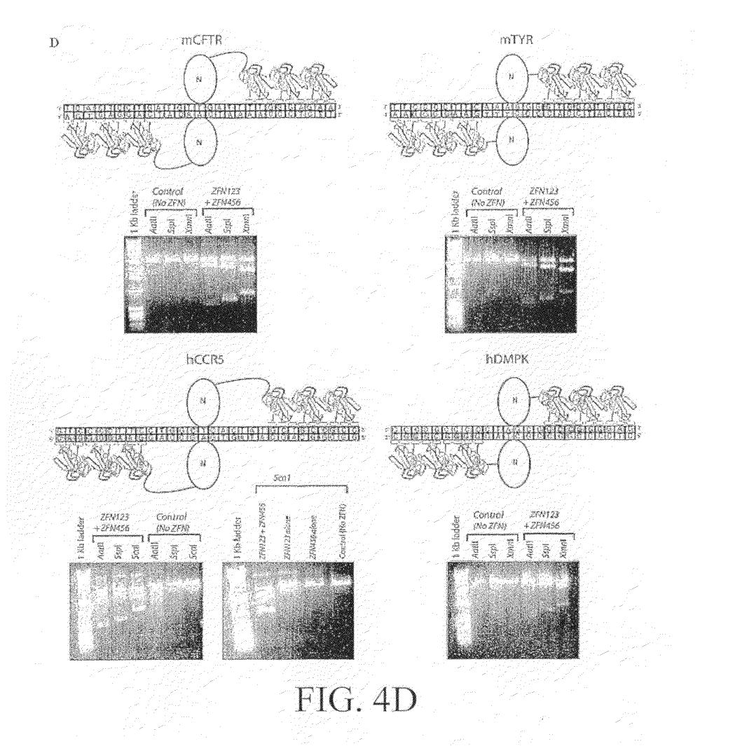

The modified in vitro transcription-translation (IVTT) assay [27] was used to rapidly screen for the sequence-specific cleavage of the engineered ZFNs. This protocol utilizes the rabbit reticulocyte IVTT system that yields sufficient amount of fusion protein product in the crude extract to study sequence-specific cleavage of substrates (FIG. 4A). Corresponding ZFN target sites were cloned into the multiple cloning sites of pUC18 to form pUC18: TS, which serve as the substrates (FIG. 4B) for the cleavage reaction. The substrates were first cut with the desired ZFNs, followed by one of the four restriction enzymes namely AatII, SspI, ScaI, or XmnI. The expected sizes of fragments resulting from such substrates cleavage are shown in FIG. 4C. The cleaved products from the ZFN digests were analyzed using agarose gel electrophoresis (FIG. 4D). The observed fragment sizes from the ZFN digests are in complete agreement with that of the expected sizes (FIG. 4C), indicating that custom-designed ZFNs find and cleave their corresponding target sites within the plasmid substrate. The agarose gel profile of the cleavage pattern for the various plasmid substrates is expected to be similar, irrespective of the ZFN targets sites encoded in them, provided the corresponding ZFN cut at their respective targets. As shown in the case of hCCR5 gel profile, the presence of both ZFN fusions, ZFN123 and ZFN 456, are needed for the substrate cleavage. Having either ZFN123 or ZFN456 alone did not cut the target site encoded within the plasmid substrate (FIG. 4D, see hCCR5 gel profile). The digests of the other substrates by their respective ZFNs also yielded similar results (data not shown).

Custom-designed ZFNs are becoming valuable tools for “gene editing” and “directed mutagenesis” of plant and mammalian genomes including the human genome. Here, we have shown the design, engineering, and rapid characterization of ZFNs that target specific sites within two mouse genes and two human genes. These are to be tested next using appropriate cell substrates and cell types for ZFN-mediated gene targeting. Several factors are critical in the design and engineering of ZFNs for gene targeting.

The first involves the ZFN target site selection within a gene of interest and availability of ZF designs needed for engineering the ZFNs. The (NNC)3 . . . (GNN)3 sites are expected to occur approximately once every 4096 bp. Since ZFNs can induce gene targeting at a distance from the site of the DSB, most if not all of the genes within the human genome are amenable to targeting by the ZFN technology. In many instances, several target sites separated by 4-12 bp are found within a gene of interest. The selection of the target site is guided by the following and in that order of importance: (1) The targets for which designs are already available in the literature are chosen. ZF designs for all GNN and ANN triplets have been published in the literature [28], [29], [30], [31] and [32]. Since ZF designs for the ANN triplets are also known, they could be incorporated in the target site selection. However, ZF designs for the ANN triplets are not as well characterized as those for the GNN triplets. While some of the ZF designs for TNN and CNN triplets are available from the literature, the complete set of ZF designs is not yet published [33]. (2) The target sites separated by 4-6 bp are highly preferred, because ZFNs without the glycine-serine linker cut these sites in a highly sequence-specific manner in vivo [21] and with high efficiency. Although not yet tested, we expect that ZFN targets separated by a 4 bp spacer will also work efficiently in cells. It must be emphasized that ZFN-mediated gene targeting efficiency falls off rapidly when the spacer is greater than 6 bp between ZFN sites; in these cases, a selection approach may be needed to identify the cells with the desired gene modification. (3) The targets closest to the mutation site are selected for ZFP design for gene editing or correction purposes. For targeted cleavage and mutagenesis by NHEJ, as in the case of hCCR5 gene, we selected the ZFN target site closest to the start codon of the CCR5 gene. In this way, we ensure deletion of most of the targeted CCR5 co-receptors and assure the production of the smallest polypeptide, if any, from the start site resulting from the premature truncation. Another consideration of importance is the availability of the ZFN designs for the adjacent triplets of the ZFN target sites, particularly for therapeutic applications; in this way, one could increase the sequence specificity of the ZFNs, as and when needed, by adding more ZF motifs to the three-finger ZFPs to form, respectively, four-, five- or even six-finger ZFNs.

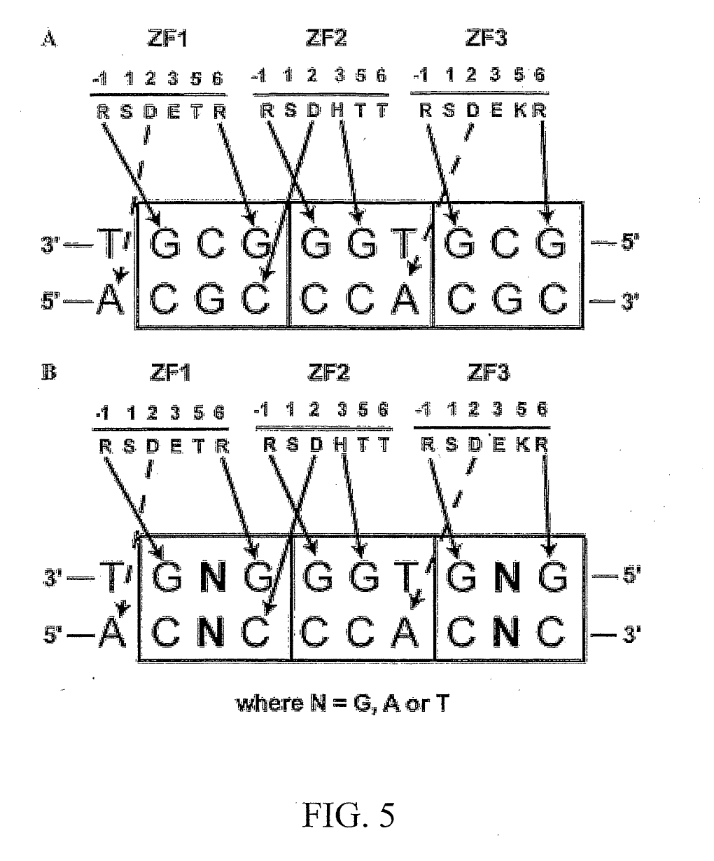

The second involves the ZFN sequence-specificity and affinity for the chosen targets within the mammalian genes. The affinity and sequence-specificity of the ZFNs to their targets are completely determined by the ZFPs, which are used to engineer them [16]. The designed ZFPs appear to have the highest affinity and sequence-specificity for their targets only when the individual ZF designs are chosen in the context of their neighboring fingers. The presence of Asp2 at position 2 of the α-helix of the preceding ZF motif promotes a cross-strand contact to a base outside the canonical triplet site, resulting in a target site overlap (FIG. 5). While this increases the affinity of the ZFPs to the target site, it also precludes the presence of a simple general recognition code for easy rational design of zinc-finger based DNA binding domains.

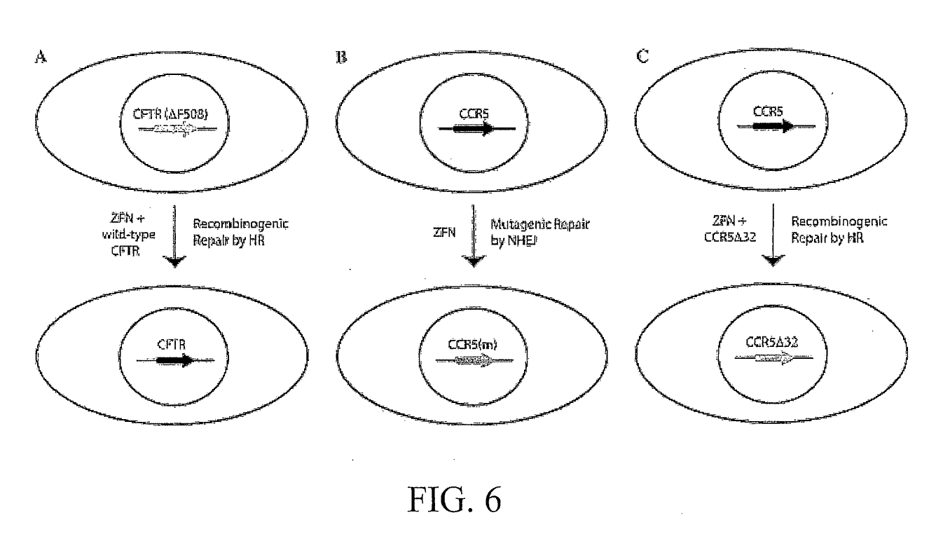

However, the results shown here and elsewhere [25] indicate that ZFNs with sufficient affinity and specificity suitable for many biological applications could be engineered by simple oligo assembly strategy. The next step for the designed ZFNs discussed in this article is to test them using appropriate mammalian cell culture studies to show that ZFN-mediated gene targeting works well in a broad range of cell types and cell substrates (FIG. 6).

The third consideration centers around the ZFNs cytotoxicity upon introduction into cells, particularly when one is interested in developing therapeutic applications. Porteus and Baltimore [23] have reported that a set of three-finger ZFNs stimulate gene targeting about 2000-fold in human cells based on the correction of a mutated GFP gene. An important finding from their work is that continued overexpression of the three-finger ZFNs in human cells was cytotoxic; as much as 75% of the targeted cells were lost due to cytotoxicity. As expected, the sequence specificity of the ZFNs appears to directly correlate with their cytotoxicity.

The individual ZF motifs usually make sequence specific contacts with only two of the bases within the cognate triplet [6] and [34] (FIG. 5A). The additional base specific cross-strand contact from the presence of Asp2 at position +2 of the α-helix of the neighboring finger that precedes the ZF motif increases the affinity and specificity of the ZF motif for its triplet subsites. If this is absent, then only two bases are generally recognized within the cognate DNA triplet, which more often than not, could result in ZF motifs recognizing other degenerate sites (FIG. 5B). Because of this, even though a set of three-finger ZFPs are expected to recognize an 18 bp target in theory, the actual recognition site is anywhere between 12 and 18 bp depending on the specificity of the chosen ZF designs for their cognate triplets. ZFNs could be engineered to be highly sequence specific by adding more fingers to the three-finger ZFPs, thereby, making them recognize a larger target DNA sequence as was done recently [24]. The ZFN target recognition was enlarged from 18 to 24 bp by using a set of four-finger ZFPs. As expected, this along with further optimization at the level of individual ZF motifs within the ZFP yielded ZFNs with high affinity and sequence specificity that were less toxic to cells. This way they achieved highly efficient and permanent modification of an endogenous gene involved in SCID in human cells. Even with the four-finger ZFNs, continued expression appears to result in cytotoxicity. Therefore, methods for regulated or controlled expression of the ZFNs within cells need to be developed for therapeutic applications.

Several other selection approaches including phage display [1], [34] and [35] are available for obtaining the desired ZFPs with high affinity from a library of mutants. However, these techniques are very laborious and cumbersome compared to the design approach particularly when the ZF designs for the target sites are already available in the literature. Recently, we have developed two simple bacterial one-hybrid systems for rapid interrogation of zinc finger-DNA interactions which might prove to be easier to perform [36].

In summary, the development of ZFNs for gene targeting by HR—the most accurate form of repair by cells—offers a precise way to site-specifically modify the plant and mammalian genomes including the human genome. ZFN-mediated gene targeting is an emerging new technology that is full of promise. Rapid design, engineering, and in vitro characterization of the ZFN cleavage specificity will greatly aid in their widespread use in various biological and biomedical applications.

### REFERENCES CITED IN EXAMPLE 1

[1] M. Moore, A. Klug and Y. Choo, Improved DNA binding specificity from polyzinc finger peptides by using strings of two-finger units, Proc. Natl. Acad. Sci. USA 98 (2001), pp. 1437-1441.
[2] R. R. Beerli and C. F. Barbas 3rd, Engineering polydactyl zinc-finger transcription factors, Nat. Biotechnol. 20 (2002), pp. 135-141.
[3] J. A. Hurt, S. A. Thibodeau, A. S. Hirsh, C. O. Pabo and J. K. Joung, Highly specific zinc finger proteins obtained by directed domain shuffling and cell-based selection, Proc. Natl. Acad. Sci. USA 100 (2003), pp. 12271-12276.
[4] L. Li, L. P. Wu and S. Chandrasegaran, Functional domains in FokI restriction endonuclease, Proc. Natl. Acad. Sci. USA 89 (1992), pp. 4275-4279.
[5] Y. G. Kim, J. Cha and S. Chandrasegaran, Hybrid restriction enzymes: zinc finger fusions to Fold cleavage domain, Proc. Natl. Acad. Sci. USA 93 (1996), pp. 1156-1160.
[6] N. P. Pavletich and C. O. Pabo, Zinc finger-DNA recognition: crystal structure of a Zif268-DNA complex at 2.1 A, Science 252 (1991), pp. 809-817.
[7] C. A. Kim and J. M. Berg, A 2.2 A resolution crystal structure of a designed zinc finger protein bound to DNA, Nat. Struct. Biol. 3 (1996), pp. 940-945.
[8] J. R. Desjarlais and J. M. Berg, Use of a zinc-finger consensus sequence framework and specificity rules to design specific DNA binding proteins, Proc. Natl. Acad. Sci. USA 90 (1993), pp. 2256-2260.
[9] Y. Shi and J. M. Berg, A direct comparison of the properties of natural and designed zinc-finger proteins, Chem. Biol. 2 (1995), pp. 83-89. SummaryPlus|Full Text+Links|PDF (2781 K)
[10] Q. Liu, D. J. Segal, J. B. Ghiara and C. F. Barbas 3rd, Design of polydactyl zinc-finger proteins for unique addressing within complex genomes, Proc. Natl. Acad. Sci. USA 94 (1997), pp. 5525-5530.
[11] R. R. Beerli, D. J. Segal, B. Dreier and C. F. Barbas 3rd, Toward controlling gene expression at will: specific regulation of the erbB-2/HER-2 promoter by using polydactyl zinc finger proteins constructed from modular building blocks, Proc. Natl. Acad. Sci. USA 95 (1998), pp. 14628-14633.
[12] J. S. Kim and C. O. Pabo, Getting a handhold on DNA: design of poly-zinc finger proteins with femtomolar dissociation constants, Proc. Natl. Acad. Sci. USA 95 (1998), pp. 2812-2817.
[13] S. Chandrasegaran and J. Smith, Chimeric restriction enzymes: what is next?, Biol. Chem. 380 (1999), pp. 841-848.
[14] K. Kandavelou, M. Mani, S. Durai and S. Chanadrasegaran, Engineering and applications of chimeric nucleases. In: A. M. Pingoud, Editor, Nucleic Acids and Molecular Biology, Springer, Berlin (2004), pp. 413-434.
[15] K. Kandavelou, M. Mani, S. Durai and S. Chandrasegaran, ‘Magic’ scissors for genome surgery, Nat. Biotechnol. 23 (2005), pp. 686-687.
[16] J. Smith, J. M. Berg and S. Chandrasegaran, A detailed study of the substrate specificity of a chimeric restriction enzyme, Nucleic Acids Res. 27 (1999), pp. 674-681.
[17] J. Bitinaite, D. A. Wah, A. K. Aggarwal and I. Schildlcraut, Fold dimerization is required for DNA cleavage, Proc. Natl. Acad. Sci. USA 95 (1998), pp. 10570-10575.
[18] D. A. Wah, J. Bitinaite, I. Schildkraut and A. K. Aggarwal, Structure of FokI has implications for DNA cleavage, Proc. Natl. Acad. Sci. USA 95 (1998), pp. 10564-10569.
[19] D. A. Wah, J. A. Hirsch, L. F. Domer, I. Schildkraut and A. K. Aggarwal, Structure of the multimodular endonuclease Fold bound to DNA, Nature 388 (1997), pp. 97-100.
[20] J. Smith, M. Bibilcova, F. G. Whitby, A. R. Reddy, S. Chandrasegaran and D. Carroll, Requirements for double-strand cleavage by chimeric restriction enzymes with zinc finger DNA-recognition domains, Nucleic Acids Res. 28 (2000), pp. 3361-3369.
[21] M. Bibikova, D. Carroll, D. J. Segal, J. K. Trautman, J. Smith, Y. G. Kim and S. Chandrasegaran, Stimulation of homologous recombination through targeted cleavage by chimeric nucleases, Mol. Cell. Biol. 21 (2001), pp. 289-297.
[22] M. Bibikova, K. Beumer, J. K. Trautman and D. Carroll, Enhancing gene targeting with designed zinc finger nucleases, Science 300 (2003), p. 764.
[23] M. H. Porteus and D. Baltimore, Chimeric nucleases stimulate gene targeting in human cells, Science 300 (2003), p. 763.
[24] F. D. Urnov, J. C. Miller, Y. L. Lee, C. M. Beausejour, J. M. Rock, S. Augustus, A. C. Jamieson, M. H. Porteus, P. D. Gregory and M. C. Holmes, Highly efficient endogenous human gene correction using designed zinc-finger nucleases, Nature 435 (2005), pp. 646-651.
[25] M. Bibikova, M. Golic, K. G. Golic and D. Carroll, Targeted chromosomal cleavage and mutagenesis in Drosophila using zinc-finger nucleases, Genetics 161 (2002), pp. 1169-1175.
[26] A. Lloyd, C. L. Plaisier, D. Carroll and G. N. Drews, Targeted mutagenesis using zinc-finger nucleases in Arabidopsis, Proc. Natl. Acad. Sci. USA 102 (2005), pp. 2232-2237.
[27] P. Ruminy, C. Derambure, S. Chandrasegaran and J. P. Salier, Long-range identification of hepatocyte nuclear factor-3 (FoxA) high and low-affinity binding sites with a chimeric nuclease, J. Mol. Biol. 310 (2001), pp. 523-535. Abstract|Full Text+Links|PDF (399 K)
[28] Q. Liu, Z. Xia, X. Zhong and C. C. Case, Validated zinc finger protein designs for all 16 GNN DNA triplet targets, J. Biol. Chem. 277 (2002), pp. 3850-3856.
[29] B. Dreier, R. R. Beerli, D. J. Segal, J. D. Flippin and C. F. Barbas 3rd, Development of zinc finger domains for recognition of the 5′-ANN-3′family of DNA sequences and their use in the construction of artificial transcription factors, J. Biol. Chem. 276 (2001), pp. 29466-29478.
[30] P. Q. Liu, E. J. Rebar, L. Zhang, Q. Liu, A. C. Jamieson, Y. Liang, H. Qi, P. X. Li, B. Chen, M. C. Mendel, X. Zhong, Y. L. Lee, S. P. Eisenberg, S. K. Spratt, C. C. Case and A. P. Wolffe, Regulation of an endogenous locus using a panel of designed zinc finger proteins targeted to accessible chromatin regions. Activation of vascular endothelial growth factor A, J. Biol. Chem. 276 (2001), pp. 11323-11334.
[31] L. Zhang, S. K. Spratt, Q. Liu, B. Johnstone, H. Qi, E. E. Raschke, A. C. Jamieson, E. J. Rebar, A. P. Wolffe and C. C. Case, Synthetic zinc finger transcription factor action at an endogenous chromosomal site. Activation of the human erythropoietin gene, J. Biol. Chem. 275 (2000), pp. 33850-33860.
[32] D. J. Segal, B. Dreier, R. R. Beerli and C. F. Barbas 3rd, Toward controlling gene expression at will: selection and design of zinc finger domains recognizing each of the 5′-GNN-3′ DNA target sequences, Proc. Natl. Acad. Sci. USA 96 (1999), pp. 2758-2763.
[33] A. C. Jamieson, J. C. Miller and C. O. Pabo, Drug discovery with engineered zinc-finger proteins, Nat. Rev. Drug Discov. 2 (2003), pp. 361-368.
[34] H. A. Greisman and C. O. Pabo, A general strategy for selecting high-affinity zinc finger proteins for diverse DNA target sites, Science 275 (1997), pp. 657-661.
[35] E. J. Rebar, H. A. Greisman and C. O. Pabo, Phage display methods for selecting zinc finger proteins with novel DNA-binding specificities, Methods Enzymol. 267 (1996), pp. 129-149.
[36] S. Durai, A. D. Bosley, A. B. Abulencia, S. Chandrasegaran, M. Ostermeier, A bacterial one-hybrid selection system for interrogating zinc finger-DNA interactions, Comb. Chem. High Throughput Screen. (accepted) (2005).

### Example 2

### Directed Mutagenesis of the CCR5 Gene in Human Cells

“Gene editing” or “directed mutagenesis” of an endogenous gene in a plant or a mammalian cell using the custom-designed ZFN entails the following steps: (1), Identify a ZFNs target site within the gene of interest. (2), Design and/or select ZFPs that recognize the target site. (3), Convert the engineered ZFPs to ZFNs. (4), Deliver the ZFN and donor DNA into cells; ZFNs are expected to direct a targeted chromosomal DSB and stimulate local HR (homology-directed repair) with the exogenously provided donor DNA. (5), Monitor for HR at the targeted chromosomal site.

HIV-1 entry into cells involves specific interactions between the viral envelope glycoprotein and two target cellular proteins, namely CD4 and a chemokine receptor. Macrophage (M)-tropic viruses require the chemokine receptor CCR5 for entry. Several studies suggest that CCR5 positive cells are the critical first targets for HIV-1 infection and that the CCR5 expression levels correlate well with disease progression. Individuals with a homozygous deletion (Δ32) in their CCR5 gene lack a functional CCR5 expression; these individuals, who are otherwise healthy, are highly protected against HIV-1 infection. Individuals who are heterozygous CCR5Δ32 reduced levels of CCR5 and their disease progression to AIDS is delayed by 1-2 years (Huang et al. 1996). Our long-term goal is to induce directed mutagenesis at the endogenous chromosomal sites of the hCCR5′ gene in primitive hematopoietic stem cells including CD34+ stem cells. Our ultimate goal is to induce targeted disruption of the chromosomal locus encoding the hCCR5 gene in hematopoietic stem cells of individuals who are at high risk for HIV infection. The autologous cells could then be used for reperfusion of the bone marrow of these individuals, thereby, malting their CD4+ lymphocytes and macrophages resistant to HIV infection.

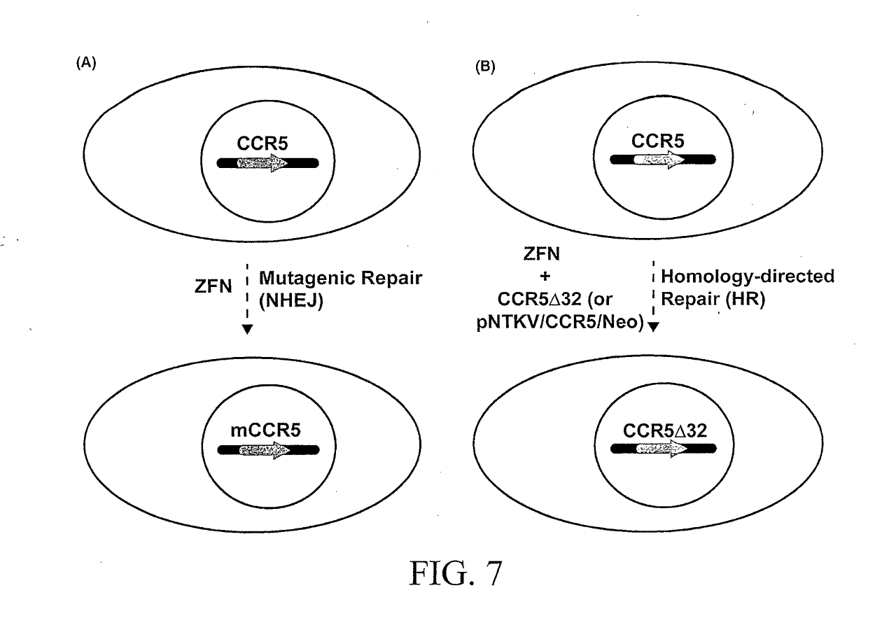

The aim here is to study the efficiency of ZFN-mediated “directed” mutagenesis of the hCCR5 gene versus ZFN cytotoxicity in human cells using the three- and four-finger ZFN respectively (FIG. 7).

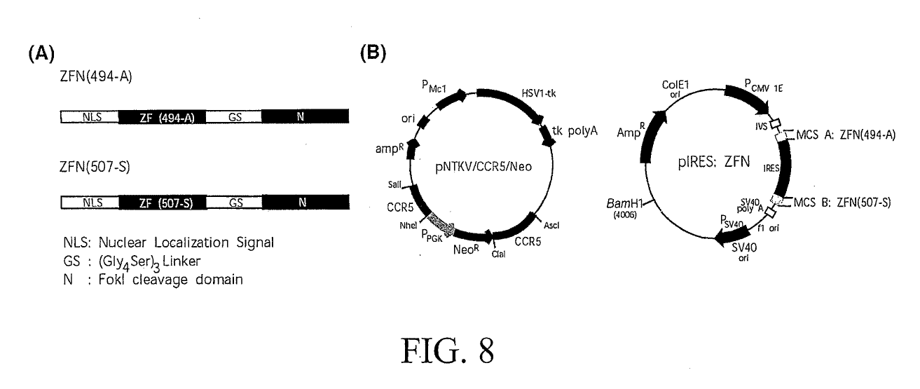

We have transferred the three-finger ZFNs that were designed to specifically target the hCCR5 gene into pIRES plasmid for use in cell culture experiments. The structure of the plasmid containing the engineered ZFNs and the plasmid substrate encoding the mutant CCR5 gene fragment as donor DNA for HR are shown in FIG. 8.

Our initial focus is to use HEK293 cells as model substrates for the engineered ZFN to show targeted disruption of the endogenous hCCR5 gene in human cells. Since the HEK293 cells do not express the CCR5 receptor on the cell surface, we have used the Flp-In T-Rex system from Invitrogen to generate a HEK293 cell line in which a single copy of CCR5 gene is under the control of tetracycline inducible promoter stably integrated within the genome.

It also has its original two copies of the endogenous hCCR5 gene. We developed this cell line for two reasons: First, these could be used to directly analyze the percentage of cells that express CCR5 before and after treatment with ZFNs to induce either mutagenic repair by NHEJ or homology-directed repair by HR in presence of exogenously added donor plasmid containing mutant CCR5 DNA or a CCR5(Δ32) DNA fragment; and second, these cells will allow a comparison of the targeting efficiency of the ZFN at two different CCR5 chromosomal loci, one of which is actively transcribed and the other that is completely silent.

(i) Generation of Flp-In-HEK293 Cell Line Expressing CCR5 Receptor

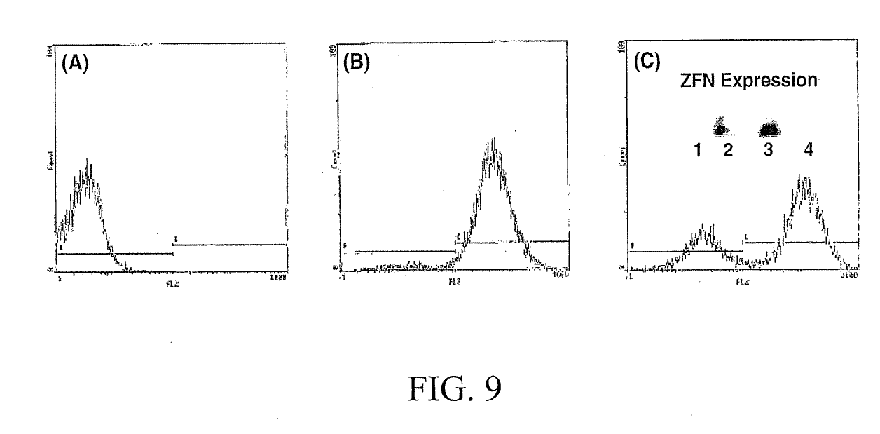

We have developed a Flp-In HEK293 cell line in which a single copy of the CCR5 gene under the control of tetracycline inducible promoter is stably integrated within the genome. It also has its original two copies of the endogenous CCR5 gene. The host cell line Flp-In HEK293 was purchased from Invitrogen. It has Flp Recombination Target site (FRT site) integrated in its genome. It also has a Tet repressor gene. The CCR5 cDNA was cloned into an expression plasmid pcDNA/FRT/TO. It also contains one FRT site, tetracycline inducible promoter and hygromycin resistance gene. The expression plasmid was co-transfected with Flp recombinase expression plasmid pOG44 into the Flp-In HEK293 cells. The Flp recombinase mediates HR between the two FRT sites and the pcDNA/FRT/TO construct is inserted into the genome at the integrated FRT site. The ATG initiation codon for the hygromycin gene is near the integrated FRT site in the genome, so the recombination event brings the ATG codon and the hygromycin gene in frame only when the integration occurs at the FRT site. Many individual clones resistant to hygromycin were screened for CCR5 expression after induction with tetracycline. The CCR5 expression was analyzed by flow cytometry (FACS), with phycoerythrin conjugated CCR5 antibody (from Pharmingen). The hygromycin resistant clones showed that 95-98% of cells express CCR5 (FIG. 9B). The CCR5 expression was also confirmed by Western blot analysis.

(ii) Targeted Disruption of CCR5 in HEK293 Flp-In Cells by Mutagenic Repair Via NHEJ

We then transfected the engineered ZFN into the CCR5 expressing HEK293 Flp-In cells. The cells were then analyzed for CCR5 expression three to four days post-transfection, when about 30 to 40% cells were negative for CCR5 expression (FIG. 9C). This follows the maximal expression of ZFN within these cells post transfection (see FIG. 9C inset). The CCR5 negative cells started to decline after four days post-transfection with ZFN and then stabilize. This decrease in the number of CCR5 negative cells suggests that continued expression of ZFN might be toxic to the cells. Methods to control the level ZFN expression in cells using regulatable promoters may be needed. These preliminary studies suggest that the engineered ZFN induce directed mutations by non-homologous end joining (NHEJ) at the CCR5 gene locus through targeted cleavage. We are in the process of sorting the cells that do not express CCR5 to analyze them for directed mutations within the CCR5 gene. The genomic DNA from some of the sorted clones that are negative for CCR5 expression will be isolated and analyzed to establish the genotype that is the disruption of the CCR5 gene at both chromosomal loci namely the FRT site where active transcription of the CCR5 gene occurs and the endogenous chromosomal site where the CCR5 gene is completely silent. We expect to recover a spectrum of mutant CCR5 clones with different genotypes.

First, the CCR5 gene surrounding the target loci will be amplified by PCR using appropriate primers specific for the FRT site and the endogenous chromosomal site respectively and then cloned into pCRII-TOPO. Individual recombinant clones will be sequenced to establish the disruption of the CCR5 gene. Second, anti-CCR5 antibody, purchased from commercial vendors, will be used to detect presence of full-length CCR5 co-receptor, if any, in the HEK293 mutant clones obtained after ZFN treatment. We expect to see only degraded fragments of the CCR5 co-receptor, if any, in the HEK293 mutant clones.

(iii) Directed Mutagenesis of the CCR5 Gene by Homology-Directed Repair Via HR

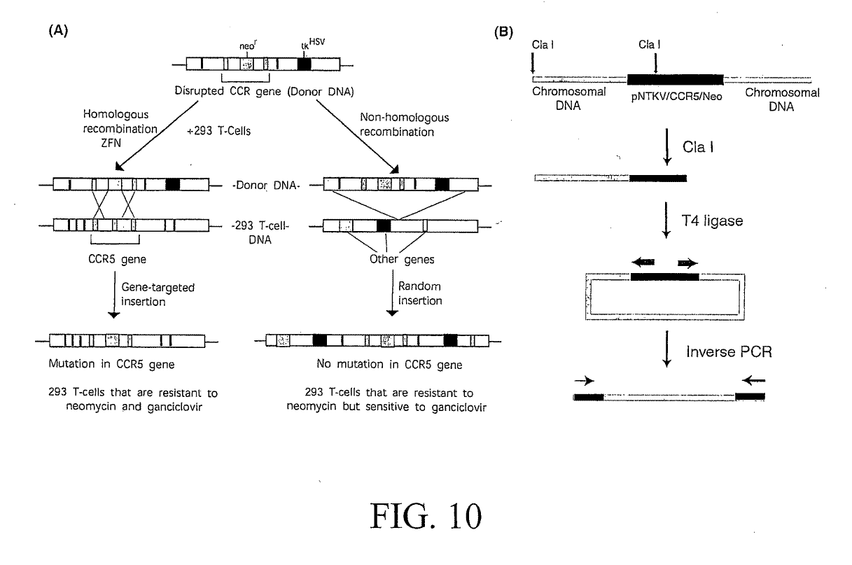

Recently, Urnov et al. (2005) have reported using ZFN-mediated gene targeting to achieve highly efficient and permanent modification of the IL2Rγ gene in human cells—a remarkable gene modification efficiency of 18% of treated cells was obtained without selection, ⅓ of which were altered on both X-chromosomes. No detectable level of random integration events using Southern blots was observed in their study. Thus, it appears that a powerful selection step may not be needed to enrich for the desired gene-modified cells. However, if it is needed, a positive-negative selection scheme (FIG. 10A) is also available for enriching CCR5 mutants during targeted disruption of hCCR5 gene using ZFNs by homology-directed repair in a HEK293 cells. In this scheme, HEK293 cells will be co-transfected with ZFN and disrupted CCR5 donor DNA with a drug marker (neomycin) (or a CCR5(Δ32) DNA fragment) and HSV-tk gene. Cells that are resistant to neomycin and ganciclovir will arise from HR while cells that are resistant to neomycin but sensitive to ganciclovir will arise from random integration events of the donor DNA. Alternatively, one could replace the neomycin gene with GFP to allow sorting of mutant recombinant clones by flow cytometry. The genomic DNA from individual mutant clones will be isolated and characterized. In presence of ZFN, we expect the recombinants arising from HR to be enriched several orders of magnitude over random integration events, based on previous studies (Porteus and Baltimore, 2003; Urnov et al. 2005). Unlike the NHEJ mutagenesis experiment, which is expected to generate a spectrum of CCR5 mutant genotypes, the homology-directed repair should result in a single homogenous mutant genotype.

We plan to use inverse PCR (IPCR) for detecting any random integration sites for the donor DNA within the mutant clones when we stimulate directed recombination by HR using ZFN and donor DNA. IPCR (Ochman et al. 1988) is routinely used for amplification and identification of sequences flanking transposable elements (FIG. 10B). We also plan to use other model substrates such as Jurkat and CD4+, CCR5+ transformed cells where the CCR5 gene can be knocked out and the infectivity experiments performed. Furthermore, the HEK293 cell line expressing CCR5 could be stably transformed with CD4+ and the CCR5 knocked out using ZFN and then the infection followed. We expect cells with the CCR5 gene mutations to show a lack of functional CCR5 expression; cells that are homozygous for these mutations should be resistant to HIV-1 infection. This can be tested by infecting with luciferase-expressing HIV-1 NL4.3 luc vector pseudo-typed with M-tropic HIV-1 envelope protein.

(2) Develop a Model System for Regulated Expression of ZFNs to Study the Efficiency of ZFN-Mediated Gene Targeting Versus ZFN Cytotoxicity in Mammalian Cells.

Even with the four-finger ZFNs, continued over-expression appears to result in cytotoxicity. Therefore, methods for regulated or transient expression of the ZFNs within cells need to be developed for therapeutic applications. We have developed a model system for regulated expression of ZFNs to study ZFN-mediated gene targeting versus ZFN cytotoxicity in mammalian cells. We have engineered ZFN that target an endogenous chromosomal site within mouse tyrosinase gene to study stable and inheritable changes in genotype and phenotype of albino monocytes. Tyrosinase is a key enzyme for melanin synthesis and pigmentation. Melanocytes that were derived from albino mice contain a homozygous point mutation TGT→TCT in the tyrosinase gene (Shibahara et al. 1990). This results in an amino acid change from Cys→Ser. Correction of this point mutation even in one allele should restore tyrosinase activity and melanin synthesis, thus changing the pigmentation of the cells. This type of correction using RNA-DNA oligonucleotides (RDO) in albino mouse melanocytes has been reported in literature (Yoon, 2002; Alexeev and Yoon, 2002, 1998; Alexeev et al. 2000).

We have developed experimental strategies for inducible expression of ZFN that target mouse tyrosinase gene to study stable and inheritable changes in genotype and phenotype of albino melanocytes. This would facilitate control of dosage as well as timing of ZFN production within the albino melanocytes. CLONTECH's Tet-Off™ Gene Expression System offers a way to achieve a regulated, high-level expression of ZFNs in mouse melanocytes. In the Tet-Off system, gene expression is turned on when tetracycline (Tc) or doxycycline (Dox, a derivative of Tc) is removed from the culture medium (Gossen and Bujard, 1992). This permits the gene expression to be tightly regulated in response to varying concentrations of Tc or Dox. Gene regulation in the Tet-Off system is highly specific and the levels of expression are very high comparable to those obtainable from strong mammalian promoters like CMV. In E. coli, the Tet repressor protein (TetR) negatively regulates the gene of the tetracycline-resistance operon on the Tn10 transposon. The TetR blocks transcription of these genes by binding to the tet operator sequences (tetO) in the absence of Tc. TetR and tetO provide the basis for Tet-Off system for use in mammalian expression systems. There are two critical components for the Tet-Off system. The first is the regulatory protein based on TetR, which is a fusion of amino acids 1-207 of TetR and the C-terminal 127 amino acids of the HSV VP16 activation domain. This fusion converts TetR from a transcriptional repressor into a transcriptional activator known as tetracycline-controlled transactivator (tTA). tTA is encoded by the Tet-Off regulator plasmid, which includes a neomycin-resistance gene to permit selection of stably transfected cells. The second critical component is the response plasmid (pTRE), which expresses the gene of interest under the control of the tetracycline-response element, TRE. The TRE consists of seven direct repeats of a 42-bp sequence containing tetO and is located just upstream of the minimal CMV promoter (P_{minCMV}).

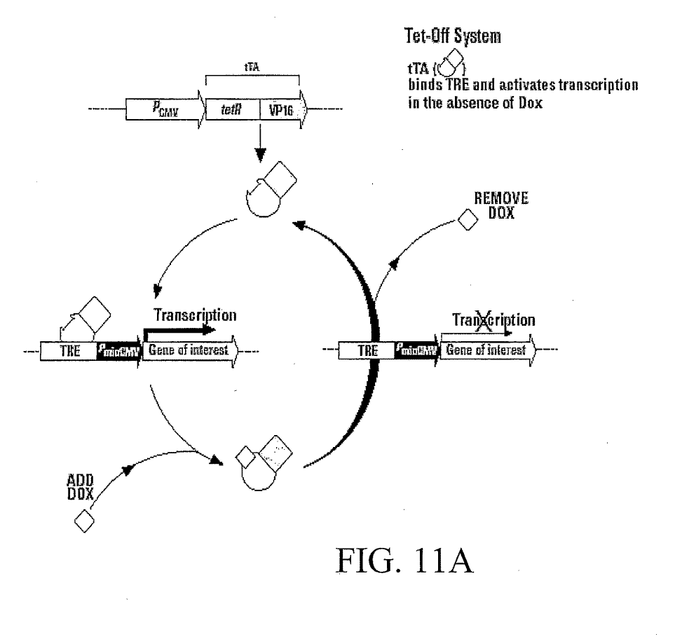

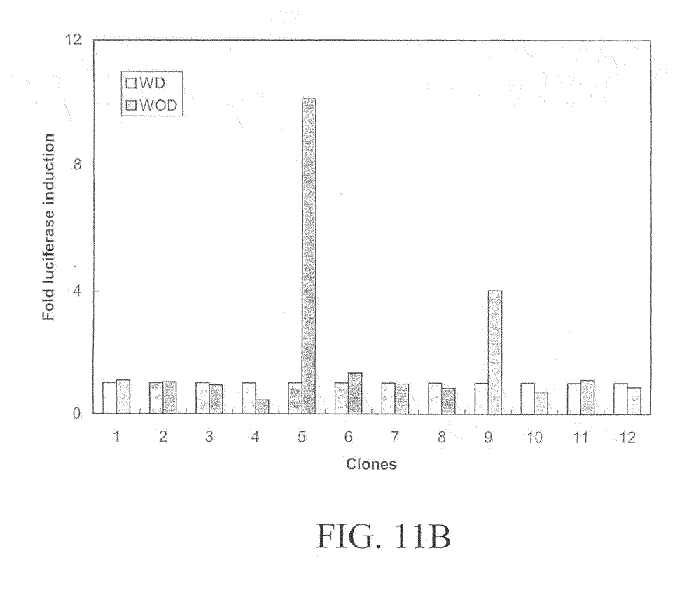

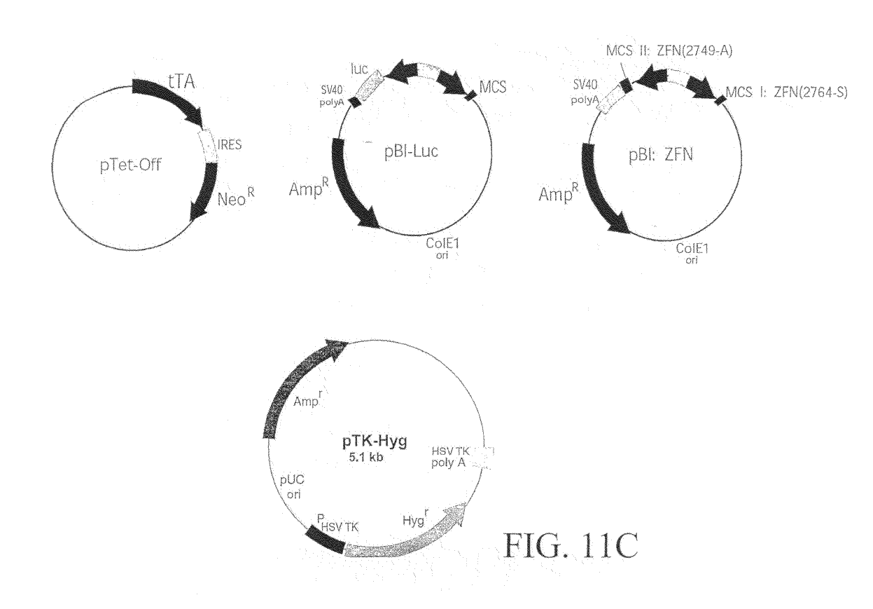

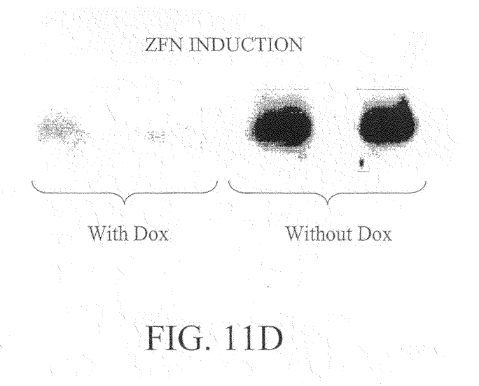

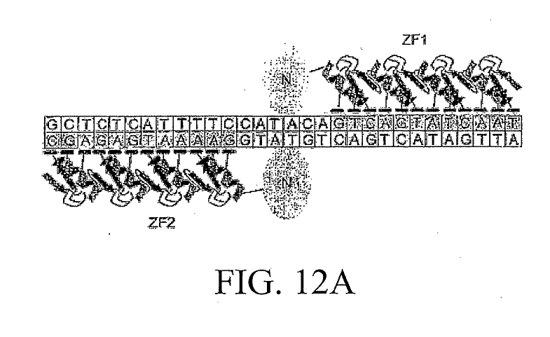

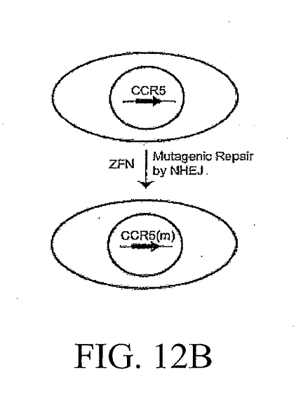

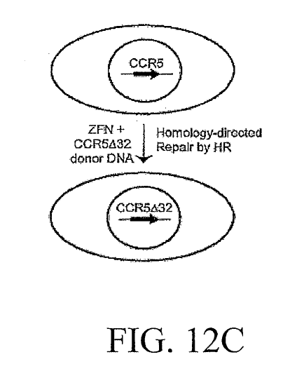

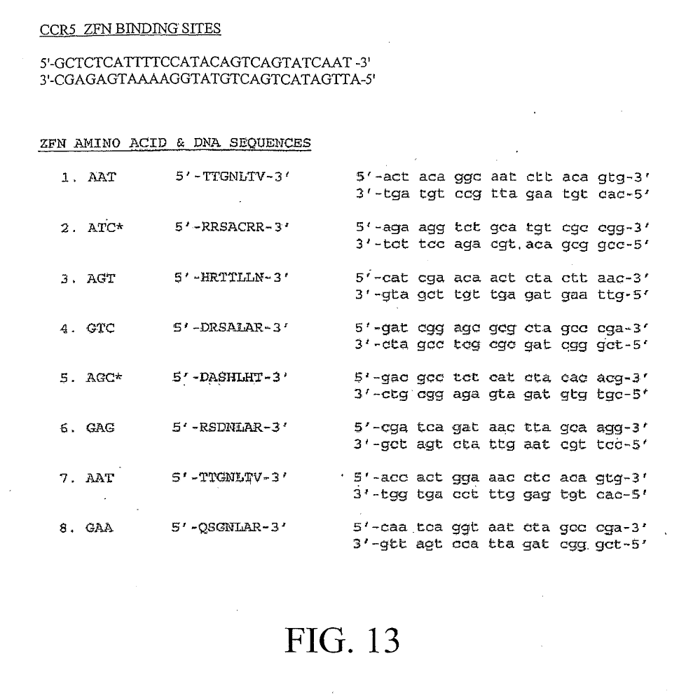

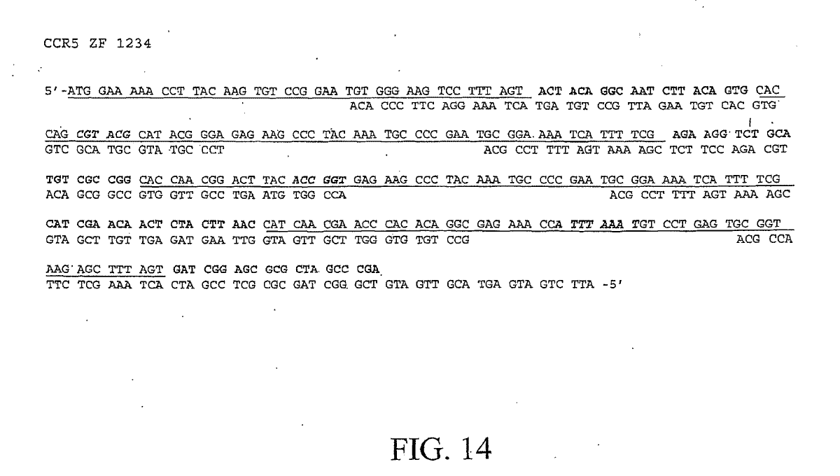

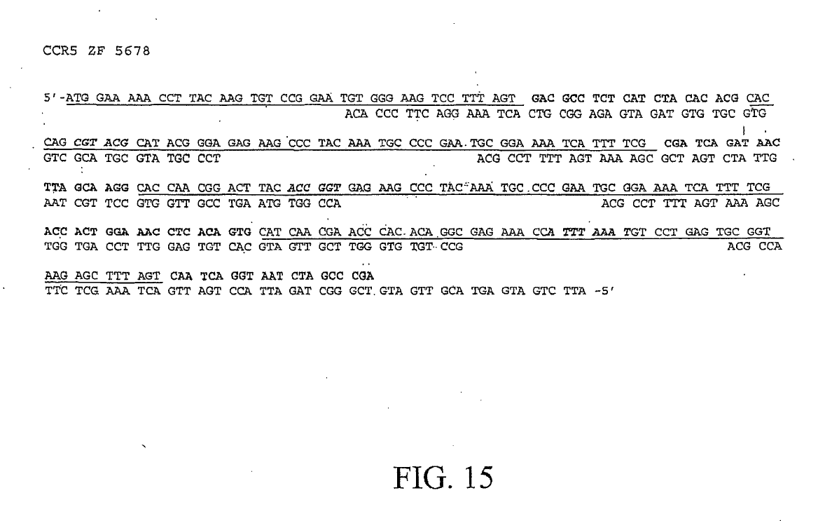

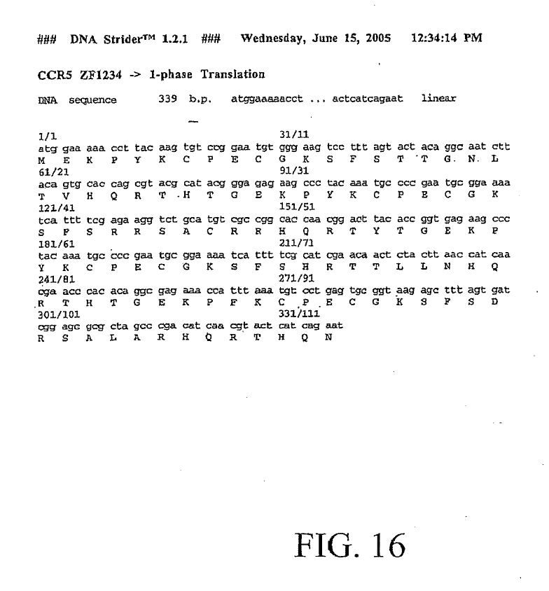

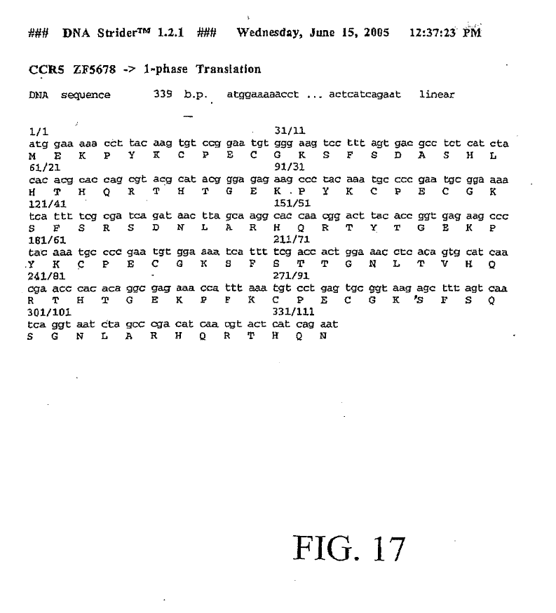

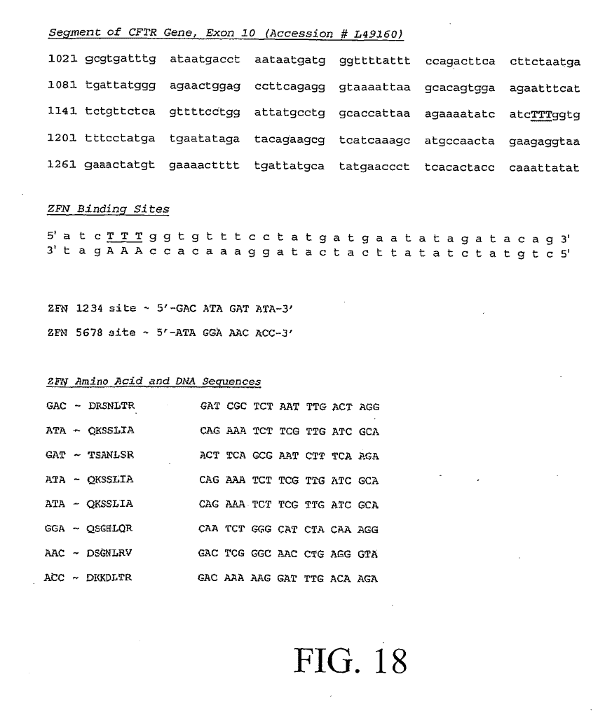

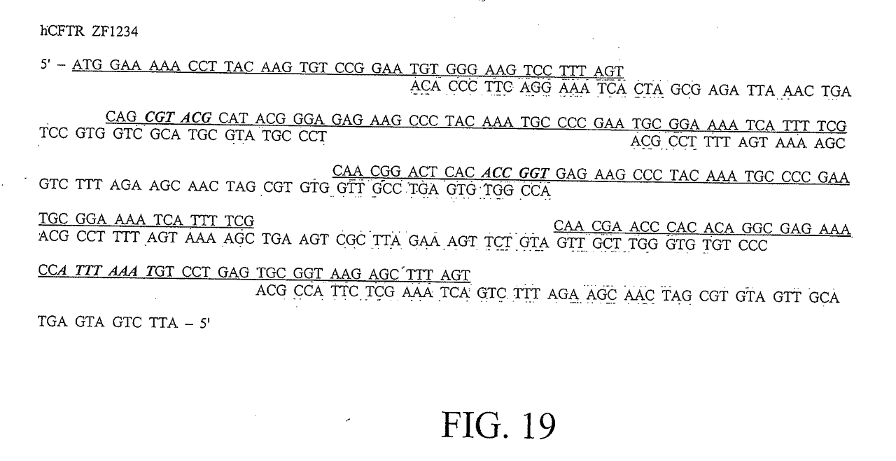

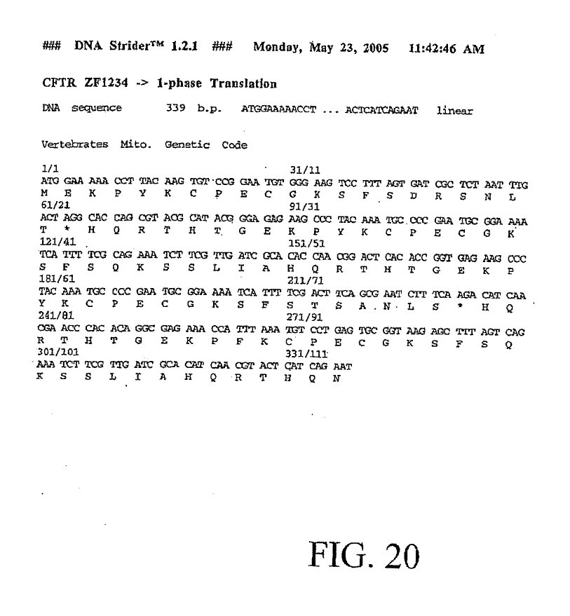

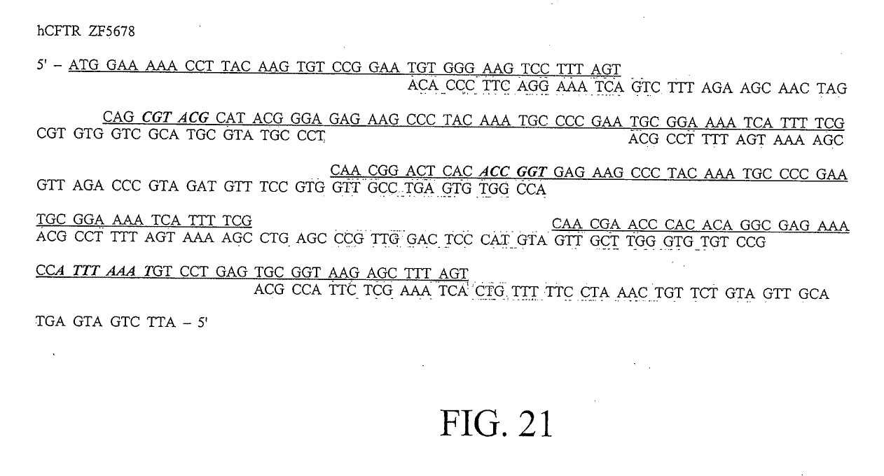

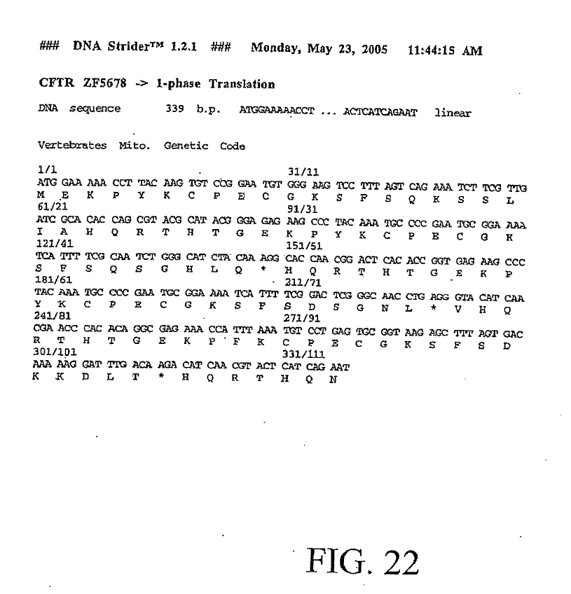

We have developed a functional Tet-Off system by creating a double-stable Tet-Off cell line of albino mouse melanocytes, which contain both the regulatory and response plasmids. When cells contain both the pTet-Off and pBI:ZFN vectors, ZFN are expressed upon binding of the tTA protein to the TRE (FIG. 11A). In absence of Tc or Dox, the tTA binds the TRE and activates transcription of ZFN. Transcription is turned off in response to Dox in a highly dose-dependent manner. First, we created stable cell lines of albino mouse melanocytes, which contain the integrated pTet-Off regulatory plasmid. Over 80 neomycin resistant individual clones were picked of which only 12 grew to confluence. These were screened for luciferase induction using a response plasmid (pBI-Luc) containing the luciferase gene. Clone #5 shows a 10-fold increase in luciferase activity in absence of Dox (FIG. 11B). Second, we transfected this cell line with the regulator plasmid pBI:ZFN and pTK-Hyg (FIG. 11C) to generate the double-stable neomycin/hygromycin resistant Tet-Off cell lines of albino mouse melanocytes. We screened over 48 hygromycin resistant clones using a gene-specific assay and identified 5 individual clones with low background and high Dox-dependent induction of ZFN. Induction of ZFN in one such representative clone is shown in FIG. 11D.

Using this double-stable Tet-Off cell line, we have initiated experiments to stimulate directed recombination in presence of donor DNA at the endogenous chromosomal locus in albino melanocytes under conditions of regulated expression of custom-designed ZFN (FIG. 11). The plan is to correct a point mutation in the TYR gene, which encodes a key enzyme for melanin synthesis and pigmentation. Following ZFN treatment of albino melanocytes in presence of donor DNA to correct the mutation, we hope to detect black-pigmented cells. The Melan A and Melan C cells were kindly provided by Drs. Alexeev and Yoon of Jefferson University. Dr. Vitali Alexeev has been collaborating with us on the mouse melanocyte cell culture experiments. He has extensively worked on this system to study targeted mutagenesis of the tyrosinase gene using RNA-DNA oligonucleotides (RDO). He will continue to serve as a collaborator/consultant for all our cell culture experiments using mouse melanocytes (see attached letter of collaboration). Mala Mani, currently a graduate student in the lab, has carefully worked out all the cell culture and transfection experimental conditions for mouse melanocyte system in consultation with Dr. Alexeev. Preliminary experiments indicate that optimal expression of ZFNs occurs between 24-48 hours post-transfection in mouse melanocytes.

Our plan is initially to use direct and simplest of assays to establish the genotype and phenotype of the converted black-pigmented clones. The methods to analyze the converted back-pigmented clones at the level of genomic sequence, protein and enzymatic activity have been well established by Alexeev and Yoon (1998). Several independent converted black-pigmented clones will be isolated from different transfection experiments. These will be subcloned 5-10 times to ensure the isolation of a black-pigmented clone from a single cell. The genomic DNA from each of the back-pigmented clones will be isolated and analyzed by restriction fragment length polymorphism (RFLP) to establish the correction of the tyrosinase gene point mutation in pigmented clones and then confirmed by DNA sequencing.

The genomic DNA from each of the converted black-pigmented clone will be subjected to PCR amplification to generate a 354 bp fragment surrounding the mutation site. The PCR product from the albino tyrosinase gene (CTAAG) should be cleaved by the restriction enzyme DdeI to yield 144, 102, 73 and 35 bp fragments. In comparison, the PCR product from homozygous wild-type tyrosinase gene (GTAAG) should result in 179, 102 and 73 bp fragments upon DdeI digestion. Thus, a 179 and a 144 bp fragment is specific for the wild type and the mutant tyrosinase gene, respectively. DNA sequencing of the 354 bp PCR fragments from the converted black-pigmented clones will be used to confirm the targeted base change (C→G). Anti-tyrosinase antibody will be used to detect the full-length tyrosinase in the pigmented clones.

We expect to see only degraded fragments in albino cells (Melan C cells) due to proteolytic cleavage of the mutant tyrosinase. Tyrosinase enzymatic activity can be detected in a non-denaturing gel, in which proteins are separated, upon incubation with L-DOPA. Oxidation of L-DOPA to melanin should result in black staining of a single band corresponding to molecular size of tyrosinase. We expect the tyrosinase activity to be detected as a single band in all converted black-pigmented cells and not in Melan-C cells since only the mature full length tyrosinase is active in L-DOPA oxidation and not other degraded fragments of tyrosinase recognized by αPEP7 polyclonal antibody in Melan-C cells. We plan to use inverse PCR (IPCR) for detecting any random integration sites for the donor DNA within the genome of the pigmented clones. IPCR (Ochman et al. 1988) is routinely used for amplification and identification of sequences flanking transposable elements (FIG. 10B). Finally, the ZFN-mediated gene correction will be compared under conditions of regulated expression of ZFNs.

### REFERENCES CITED IN EXAMPLE 2

Alexeev V, Igoucheva O, Domashenko A, Cotsarelis G, Yoon K (2000) Localized in vivo genotypic and phenotypic correction of the albino mutation in skin by RNA-DNA oligonucleotide. Nat Biotech 18: 43-47.
Alexeev V, Yoon K (1998) Stable and inheritable changes in genotype and phenotype of albino melanocytes induced by an RNA-DNA oligonucleotide. Nat Biotech 16: 1343-1346.
Capecchi MR (1989) Altering the genome by homologous recombination. Science 244:1288-1292.
Gossen M, Bujard H (1992) Tight control of gene expression in mammalian cells by tetracycline Responsive promoters. Proc. Natl. Acad. Sci. USA 89: 5547-5551.
Ochman H, Gerber A S, Hartl D L (1988) Genetic applications of an inverse polymerase chain reaction. Genetics 120: 621-623.
Porteus M H, Baltimore D (2003) Chimeric nucleases stimulate gene targeting inhuman cells. Science 300: 763.
Shibahara S, Okinaga S, Tomkta Y, Takeda A, Yamamoto H, Sato M, Takeuchi T (1990) A point mutation in the tyrosinase gene of BALB/c albino mouse causing the cysteine-serine substitution at position 85. Eur J Biochem 189: 455-461.
Urnov F D, Miller J C, Lee Y L, Beausejour C M, Rock J M, Augustus S, Jamieson A C, Porteus M H, Gregory P D, Holmes M C. (2005) Highly efficient endogenous human gene correction using designed zinc-finger nucleases. Nature 435, 646-651.
Yoon K (2002) Expectations and reality in gene repair. Nat Biotechnol. 20: 1197-1198.

All patents, patent applications (including 60/702,260) and publications mentioned herein are hereby incorporated by reference, in their entireties, for all purposes.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

1. A method of cleaving a gene of interest in a cell, the method comprising:
providing a fusion protein comprising a zinc finger binding domain and a Fok I cleavage domain, wherein the zinc finger binding domain binds to a target site in the gene of interest; and
contacting the cell with the fusion protein under conditions such that the gene of interest is cleaved.

2. The method of claim 1, further comprising contacting the cell with a polynucleotide, wherein the polynucleotide replaces sequences in the cleaved gene of interest.

3. The method of claim 2, wherein the replaced sequences of the gene of interest comprise at least one mutation associated with a disease or condition mediated by a mutant form of the gene of interest.

4. The method of claim 1, wherein the gene of interest is CFTR, the zinc finger binding domain binds to a target site in the CFTR gene, and the CFTR gene is cleaved.

5. The method of claim 4, further comprising the step of contacting the cell with a polynucleotide, wherein the polynucleotide replaces sequences in the cleaved CFTR gene.

6. The method of claim 5, wherein the replaced sequences of the CFTR gene comprise at least one mutation associated with cystic fibrosis.

7. The method of claim 4, wherein the zinc finger binding domain comprises, as a recognition region, one of the six 7 amino acid sequences shown for mCFTR in Table 1.

8. The method of claim 7, wherein the zinc finger binding domain comprises three zinc fingers, wherein the recognition region of each of the three zinc fingers is ZF1, ZF2 or ZF3.

9. The method of claim 7, wherein the zinc finger binding domain comprises three zinc fingers, wherein the recognition region of each of the three zinc fingers is ZF4, ZF5 or ZF6.

10. A composition useful for disrupting a CFTR gene in a cell, comprising an engineered fusion protein which comprises a zinc finger binding domain to bind the CFTR target sequence and a FokI cleavage domain, wherein the fusion protein binds to and cleaves the CFTR gene.

11. The composition of claim 10, wherein the zinc finger binding domain comprises, as a recognition region, one of the six 7 amino acid sequences shown for mCFTR in Table 1.

12. The composition of claim 11, wherein the zinc finger binding domain comprises three zinc fingers, wherein the recognition region of each of the three zinc fingers is ZF1, ZF2 or ZF3.

13. The composition of claim 11, wherein the zinc finger binding domain comprises three zinc fingers, wherein the recognition region of each of the three zinc fingers is ZF4, ZF5 or ZF6.

14. The method of claim 1, wherein the gene of interest is DMPK, the zinc finger binding domain binds to a target site in the DMPK gene, and the DMPK gene is cleaved.

15. The method of claim 14, further comprising the step of contacting the cell with a polynucleotide, wherein the polynucleotide replaces sequences in the cleaved DMPK gene.

16. The method of claim 15, wherein the replaced sequences of the DMPK gene comprise at least one mutation associated with myotonic dystrophy.

17. The method of claim 14, wherein the zinc finger binding domain comprises, as a recognition region, one of the six 7 amino acid sequences shown for hDMPK in Table 1.

18. The method of claim 17, wherein the zinc finger binding domain comprises three zinc fingers, wherein the recognition region of each of the three zinc fingers is ZF1, ZF2 or ZF3.

19. The method of claim 17, wherein the zinc finger binding domain comprises three zinc fingers, wherein the recognition region of each of the three zinc fingers is ZF4, ZF5 or ZF6.

20. A composition useful for disrupting a DMPK gene in a cell, comprising an engineered fusion protein which comprises a zinc finger binding domain to bind the DMPK target sequence and a FokI cleavage domain, wherein the fusion protein binds to and cleaves the DMPK gene.

21. The composition of claim 20, further wherein the zinc finger binding domain comprises, as a recognition region, one of the six 7 amino acid sequences shown for hDMPK in Table 1.

22. The composition of claim 21, wherein the zinc finger binding domain comprises three zinc fingers, wherein the recognition region of each of the three zinc fingers is ZF1, ZF2 or ZF3.

23. The composition of claim 21, wherein the zinc finger binding domain comprises three zinc fingers, wherein the recognition region of each of the three zinc fingers is ZF4, ZF5 or ZF6.

24. The method of claim 1, wherein the gene of interest is CCR5, the zinc finger binding domain binds to a target site in the CCR5 gene, and the CCR5 gene is cleaved.

25. The method of claim 24, further comprising the step of contacting the cell with a polynucleotide, wherein the polynucleotide replaces sequences in the cleaved CCR5 gene.

26. The method of claim 25, wherein the replaced or replacing sequences comprise at least one mutation associated with CCR5.

27. The method of claim 24, further wherein the CCR5 gene after cleavage is repaired by non-homologous end-joining in the cell to give rise to a CCR5 gene mutation that inactivates the CCR5 receptor.

28. The method of claim 25, wherein the replacing sequences comprise the CCR5delta 32 mutation, thereby inactivating the CCR5 receptor.

29. The method of claim 24, further wherein the CCR5 chromosomal gene locus after cleavage serves as a “safe harbor” site within the human genome for introducing and ectopically expressing other human genes as transgenes in human cell types for human therapeutics.

30. The method of claim 25, wherein the replacing sequences encode a therapeutic protein or marker gene.

31. The method of claim 30, wherein the marker gene is neomycin or green fluorescent protein (GFP).

32. The method of claim 24, wherein the zinc finger binding domain comprises, as a recognition region, one of the six 7 amino acid sequences shown for hCCR5 in Table 1.

33. The method of claim 32, wherein the zinc finger binding domain comprises three zinc fingers, wherein the recognition region of each of the three zinc fingers is ZF1, ZF2 or ZF3.

34. The method of claim 32, wherein the zinc finger binding domain comprises three zinc fingers, wherein the recognition region of each of the three zinc fingers is ZF4, ZF5 or ZF6.

35. The method of claim 24, wherein the cell is a human primary cell, a human adult stem cell, a human embryonic stem cell or a human hematopoietic stem cell.

36. A composition useful for disrupting a CCR5 gene in a cell, comprising an engineered fusion protein which comprises a zinc finger binding domain to bind the CCR5 target sequence and a FokI cleavage domain, wherein the fusion protein binds to and cleaves the CCR5 gene.

37. The composition of claim 36, wherein the zinc finger binding domain comprises, as a recognition region, one of the six 7 amino acid sequences shown for hCCR5 in Table 1.

38. The composition of claim 37, wherein the zinc finger binding domain comprises three zinc fingers, wherein the recognition region of each of the three zinc fingers is ZF1, ZF2 or ZF3.

39. The composition of claim 37, wherein the zinc finger binding domain comprises three zinc fingers, wherein the recognition region of each of the three zinc fingers is ZF4, ZF5 or ZF6.

40. The method of claim 1, wherein the gene of interest is TYR, the zinc finger binding domain binds to a target site in the TYR gene, and the TYR gene is cleaved.

41. The method of claim 40, further comprising the step of contacting the cell with a polynucleotide, wherein the polynucleotide replaces sequences in the cleaved TYR gene.

42. The method of claim 41, wherein the replaced sequences of the TYR gene comprise at least one mutation associated with tyrosinase enzyme activity.

43. The method of claim 40, wherein the zinc finger binding domain comprises, as a recognition region, one of the six 7 amino acid sequences shown for mTYR in Table 1.

44. The method of claim 43, wherein the zinc finger binding domain comprises three zinc fingers, wherein the recognition region of each of the three zinc fingers is ZF1, ZF2 or ZF3.

45. The method of claim 43, wherein the zinc finger binding domain comprises three zinc fingers, wherein the recognition region of each of the three zinc fingers is ZF4, ZF5 or ZF6.

46. The method of claim 40, wherein the cell is a human melanocyte or a human stem cell.

47. A composition useful for disrupting a TYR gene in a cell, comprising an engineered fusion protein which comprises a zinc finger binding domain to bind the TYR target sequence and a FokI cleavage domain, wherein the fusion protein binds to and cleaves the TYR gene.

48. The composition of claim 47, wherein the zinc finger binding domain comprises, as a recognition region, one of the six 7 amino acid sequences shown for mTYR in Table 1.

49. The composition of claim 48, wherein the zinc finger binding domain comprises three zinc fingers, wherein the recognition region of each of the three zinc fingers is ZF1, ZF2 or ZF3.

50. The composition of claim 48, wherein the zinc finger binding domain comprises three zinc fingers, wherein the recognition region of each of the three zinc fingers is ZF4, ZF5 or ZF6.

51. The method of claim 1, wherein the gene of interest is beta globin, the zinc finger binding domain binds to a target site in the beta globin gene, and the beta globin gene is cleaved.

52. The method of claim 51, further comprising the step of contacting the cell with a polynucleotide, wherein the polynucleotide replaces sequences in the cleaved beta globin gene.

53. The method of claim 52, wherein the replaced sequences of the beta globin gene comprise at least one mutation associated with sickle cell anemia.

54. The method of claim 1, wherein the gene is a human gene.

55. The method of claim 1, wherein the cell is a human cell.

56. The composition of claim 10, wherein the gene is a human gene.
